(19) United States
(12) Patent Application Publication
(10) Pub. No.: US 2008/0124249 A1
(43) Pub. Date: May 29, 2008

(54) Apparatus For Recovering Specific Substance And Nucleic Acid Extracting Apparatus Using The Same

(76) Inventor: Toshihiro Mori, Saitama (JP)

Correspondence Address: BIRCH STEWART KOLASCH & BIRCH, PO BOX 747, FALLS CHURCH, VA, 22040-0747, US

(21) Appl. No.: 11/793,331

(22) PCT Filed: Jan. 31, 2006
(86) PCT No.: PCT/JP06/01928

§ 371 Date: Jun. 19, 2007

(30) Foreign Application Priority Data
Jan. 31, 2005 (JP) 2005-022676

(51) Int. Cl. B01L 11/00; C12M 1/12

(52) U.S. Cl. 422/101; 435/297.2

(57) ABSTRACT

With the nucleic acid extracting apparatus 100, which uses the apparatus for recovering a specific substance, a sample solution S is injected into a cartridge 11 which is provided with a filter member 11b, and pressure is added to adsorb a specific substance on the filter member 11b, and a recovering solution R is injected into the cartridge 11, and pressure is added to recover the specific substance adsorbed on the filter member 11b with the recovering solution R. In addition, the apparatus 100 is provided with a pressurized air supply means 4, a pressure detection means 46 and a means 70 for deciding a subject to be treated which decides if or not it is a cartridge which is a subject to be treated, for which recovery is carried out for the specific substance, by the pressure detected by the pressure detection means 46 for when the pressurized air is introduced into the cartridge by the pressurized air supply means.

### TECHNICAL FIELD

The present invention relates to an apparatus for recovering a specific substance, which automatically extracts a specific substance, for example, nucleic acid and the like in a sample solution, using a cartridge which is provided with a filter member.

### BACKGROUND ART

As a conventional method for extracting a specific substance, for example, nucleic acid, mention may be made of a method by centrifuge, a method by magnetic beads, a method by filter and the like.

As a nucleic acid extracting apparatus using a filter for extracting nucleic acid, which is an example of the specific substance, a device has been suggested, wherein a large number filer tubes containing a filter are set on a rack, and a sample solution is separately injected thereto, the circumference of the bottom of the rack is sealed with an air chamber through a sealing material to reduce the pressure of the inside, all the filter tubes are sucked from the discharge side at a time to pass the sample solution, and thereby adsorb nucleic acid on the filter, and then, a washing solution and an eluting solution are injected, and sucked under reduced pressure similarly to wash and elute them (for example, see Japanese Patent No. 2832586).

### DISCLOSURE OF THE INVENTION

However, the conventional automatic extraction apparatus as described above has a large size and thus it is suitable for analyzing large amount of test samples. Therefore, when the number of the test samples is small and the analysis frequency is low, it has problems that it is not suitable because of high cost, and the treatment efficiency is lowered.

In addition, for such apparatus, it is desired that the treatment is done in a short time with high efficiency without incidence of contamination, and further that the apparatus is minimized, etc. However, with reference to Japanese Patent No. 2832586, there are the following problems.

When the properties of each sample solution are different from each other such as the collected whole blood, with an apparatus which sucks the whole at a time as in Japanese Patent No. 2832586, a part of the filter tubes are completed for suction, which leads to no resistance. Then, the reduced pressure exerted to other filter tubes becomes small, which may induce that the treatment for a sample solution having high viscosity is not completed. If the capacity of reduced pressure increases, it may be an obstacle when minimization of an apparatus is intended, and it takes time to work reduced pressure since the volume of reduced pressure increases. In addition, it is difficult to detect that all the solution is discharged, and time set is long, which may be an obstacle for improvement of the treatment efficiency. On the other hand, for a sample solution having low viscosity, the problem is that the solution is discharged strongly from the filter tube, bubble droplets adhere to the adjacent filter tube and rack, which leads to contamination, and thereby leads to reduced accuracy.

The present applicants have conducted extensive studies to solve the above described problems, and as results, they have completed a novel nucleic acid extracting apparatus which is one example of an apparatus for recovering a specific substance in a sample solution. This nucleic acid extracting apparatus is an apparatus wherein using a sample solution containing nucleic acid, nucleic acid in the sampled solution containing nucleic acid, is adsorbed on nucleic acid-adsorbing porous membrane by a nucleic acid extraction cartridge containing a nucleic acid-adsorbing porous membrane (filter member) in a container (hereinafter, referred to as the cartridge), and a pressure-generating apparatus, and then it is separated and purified after washing, etc.

The novel nucleic acid extracting apparatus is provided with plural groups of a cartridge, which is equipped with a nucleic acid-adsorbing porous membrane, and which a sample solution containing nucleic acid is injected into, a waste liquor container and a recovering container, which are placed under the cartridge in correspondence to each cartridge. With the nucleic acid extracting apparatus, a washing solution W and a recovering solution R are separately injected into the cartridge, and at the same time, the pressurized air is introduced into the cartridge by the pressurized air supply means, to recover the recovery solution containing nucleic acid from the sample solution into the recovering container. The plural groups of the cartridge, the waste liquor container and the recovering container are placed as aligned, and for each cartridge, the above extraction procedures are carried out sequentially.

As shown in FIG. 16(a), for a cartridge 11, which is used in the novel nucleic acid extracting apparatus, and into which a sample solution containing a specific substance is injected, nucleic acid-adsorbing porous membrane (filter member) 11b is held at the bottom of a cylindrical main body 11a of which the top is open, the lower part is formed in the lot shape by the nucleic acid-adsorbing porous membrane 11b of the cylindrical main body 11a, a discharge unit 11c of a tubular nozzle shape is formed as projected in a prescribed length at the lower central part, and longitudinal projections 11d are formed on both sides of the lateral part of the cylindrical main body 11a. From an upper opening 11e of the cartridge 11, a sample solution, a washing solution and a recovering solution as described below are separately injected, and then pressurized air is introduced from the upper opening 11e, and each solution is flew down and discharged into a waste liquor container 12 or a recovering container 13 as described below through the nucleic acid-adsorbing porous membrane 11b.

Furthermore, in case of the schematic illustration, the cylindrical main body 11a has a structure such that it is divided into the upper part and the lower part, and the two parts fit to each other. In addition, the upper opening 11e has an inclined surface 11f, which is made by cutting the inner circumferential surface in the taper shape as shown in a XVIB-XVIB section in FIG. 16(b). This inclined surface 11f is formed to correspond significantly to the inclined outer circumferential surface of the top of the pressurizing nozzle of the pressurized air supply means (not shown).

An outline of the nucleic acid extraction process by the nucleic acid extracting apparatus will be explained below.

The nucleic acid extracting apparatus basically performs nucleic acid extraction by the extraction process as shown in FIGS. 17(a) to (g). First, in the process (a) of FIG. 17(a), a sample solution S containing nucleic acid which is treated as dissolved, is injected into a cartridge 11, which is located on a waste liquor container 12. Next, in the process (b), the pressurized air is introduced into the cartridge 11 to add pressure, and the sample solution S is passed through a nucleic acid-adsorbing porous membrane 11b, whereby nucleic acid is adsorbed on the nucleic acid-adsorbing porous membrane 11b, and the passed liquid components are discharged into the waste liquor container 12.

Next, in the process (c), a washing solution W is automatically injected into the cartridge 11, and in the process (d), the pressurized air is introduced into the cartridge 11 to add pressure, and with DNA held on the nucleic acid-adsorbing porous membrane 11b, washing out and removing for other impurities are carried out, and the passed washing solution W is discharged into the waste liquor container 12. Such the processes (c) and (d) may be repeated plural times.

Then, in the process (e), a waste liquor container 12 below the cartridge 11, is exchanged with a recovering container 13, and then in the process (f), a recovering solution R is automatically injected into the cartridge 11. In the process (g), the pressurized air is introduced into the cartridge 11 to add pressure, thereby weaken the binding force between the nucleic acid-adsorbing porous membrane 11b and nucleic acid, and separate the adsorbed nucleic acid, and the recovering solution R containing nucleic acid is discharged and recovered into the recovering container 13.

The nucleic acid-adsorbing porous membrane 11b in the cartridge 11 is basically a porous body through which nucleic acid can pass, and is constituted such that the surface thereof has a property of adsorbing nucleic acid in a sample solution with chemical binding force, the adsorption is maintained in washing by a washing solution, and the adsorption force of nucleic acid is weakened and nucleic acid is separated in recovering by a recovering solution.

However, a nucleic acid extracting apparatus which conducts the above processes automatically had the following problems.

A plurality of cartridges 11 can be equipped as aligned in a cartridge holder as to conduct efficient nucleic acid extraction. When the number of the sample solutions to be extracted for nucleic acid is small, and the cartridges 11 are equipped in less number than that of the cartridges that can be equipped (i.e., the cartridge holder has a part where no cartridge 11 is equipped), or a cartridge 11 is equipped which is lack of a nucleic acid-adsorbing porous membrane 11b for any reason, or there is a cartridge 11 into which no sample solution is injected by mistake, the nucleic acid extracting apparatus cannot detect these disorders, and it conducts extraction processes of nucleic acid sequentially as if all of the cartridges 11 are equipped normally.

As described above, if extraction procedure is conducted at a spot where extraction procedure is not needed or substantially useless originally such as the case of no equipment of the cartridge 11, lack of the nucleic acid-adsorbing porous membrane 11b and no injection of the sample solution, this is time loss and the work efficiency is remarkably reduced, which prevents improvement of extraction performance. In addition, if a washing solution W and a recovering solution R are separately injected into such spot, the washing solution W and the recovering solution R get dropped in the main body of the apparatus, which leads to a problem of contamination of the inside of the apparatus. Such problems can be avoided if a waste liquor container 12 and a recovering container 13 are also placed on a spot which does not need them originally to contain the dropped, useless washing solution W and recovering solution R. However, in case of no equipment of the cartridge 11, the washing solution W and the recovering solution R are dropped from an injection nozzle that are placed pretty upper than the waste liquor container 12 and the recovering container 13, so the droplets of the solutions fly, which may possibly contaminate the surroundings. In addition, the waste liquor container 12 and recovering container 13 were discarded without being used properly, which was one of the causes to increase the cost of nucleic acid extraction.

In light of such problems, the object of the present invention is to provide an apparatus for recovering a specific substance which can conduct recovery of a specific substance in a sample solution, wherein the apparatus has high efficiency, and is simple and quick, and excellent in automation suitability, and has high reproductivity, and a nucleic acid extracting apparatus using the same.

Specifically, the constitution of the present invention will be explained below.

(1) An apparatus for recovering a specific substance in a sample solution, which comprises:

a cartridge holder that can contain a plurality of cartridges; and

a plurality of cartridges each having a filter member, and held by the cartridge holder,

wherein a sample solution is injected into the cartridge held by the cartridge holder, and a pressure is applied to adsorb a specific substance in the sample solution on the filter member, and a recovering solution is injected into the cartridge, and a pressure is applied to recover the specific substance adsorbed on the filter member with the recovering solution, and

wherein the apparatus further comprises:

a pressurized air supply means that introduces a pressurized air from a pressurizing nozzle into the cartridge;

a pressure detecting means that detects a pressure within the cartridge; and

a means for deciding a subject to be treated that decides whether or not the cartridge is a cartridge which is a subject to be treated for recovering the specific substance, based on the pressure detected by the pressure detecting means when the pressurized air is introduced into the cartridge by the pressurized air supply means.

According to this apparatus for recovering a specific substance, the means for deciding a subject to be treated decides if or not it is a cartridge which is a subject to be treated, for which recovery is carried out for the specific substance, based on the pressure detected by the pressure detection means when the pressurized air is introduced into the cartridge, and then extraction processes for a specific substance is carried out only for the cartridge which is a subject to be treated. According to this, it is possible to try the tact-up of extraction processes, and improve operation efficiency of the apparatus for recovering a specific substance.

(2) The apparatus for recovering a specific substance in a sample solution as described in (1) above,

wherein the means for deciding a subject to be treated excludes a cartridge from the subject to be treated when a peak value of the pressure within the cartridge, which is detected by the pressure detection means, is lower than a preset value.

According to this apparatus for recovering a specific substance, if a peak value of the pressure within the cartridge, which is detected by the pressure detection means, is lower than a preset value, the cartridge is excluded from the subject to be treated, which makes it possible to stop the following extraction processes for the cartridge. In addition, according to this, it is possible to conduct efficient extraction.

(3) The apparatus for recovering a specific substance in a sample solution as described in (1) above,

wherein the means for deciding a subject to be treated excludes a cartridge from the subject to be treated when an integral value for a certain time of the pressure within the cartridge, which is detected by the pressure detection means, is lower than a preset value.

According to this apparatus for recovering a specific substance, if an integral value for a certain time of the pressure within the cartridge, which is detected by the pressure detection means, is lower than a preset value, the cartridge is excluded from the subject to be treated, which makes it possible to stop extraction processes for the cartridge after this. In addition, according to this, it is possible to conduct efficient extraction operation.

(4) The apparatus for recovering a specific substance in a sample solution as described in any one of (1) to (3) above,

wherein the pressurizing nozzle of the pressurized air supply means is supported movably along a direction of a cartridge mounting of the plurality of cartridges contained in the cartridge holder.

According to this apparatus for recovering a specific substance, the pressurizing nozzle is supported movably along the direction of the cartridge mounting, so the pressurized air is supplied sequentially to each one of a plurality of cartridges aligned to decide if or not the cartridge is a cartridge which is a subject to be treated, which makes it possible to conduct proper extraction processes.

(5) The apparatus for recovering a specific substance in a sample solution as described in (4) above,

wherein the pressurizing nozzle and at least a separate injection nozzle that discharges the recovering solution, are installed as integrated with a movable body.

According to this apparatus for recovering a specific substance, the pressurizing nozzle and the injection nozzle are installed as integrated onto the movable body, so it is possible to conduct efficient supplying of pressurized air and injection of a recovering solution.

(6) The apparatus for recovering a specific substance in a sample solution as described in any one of (1) to (5) above,

wherein the filter member is one of a porous membrane, a non-woven fabric and a textile.

According to this apparatus for recovering a specific substance, the filter member is any one of a porous membrane, a non-woven fabric, or a textile, so it is possible to use a filter member which has optimal properties depending on a specific substance to be extracted. According to this, it is possible to conduct efficient recovery in correspondence to various kinds of specific substances only with changing the filter member.

(7) The apparatus for recovering a specific substance in a sample solution as described in any one of (1) to (6) above,

wherein the specific substance is a living body-derived substance or a biological material.

According to this apparatus for recovering a specific substance, it is possible to recover a living body-derived substance or a biological material.

(8) An apparatus for recovering a specific substance in a sample solution as described in any one of (1) to (7) above, which is a nucleic acid extracting apparatus,

wherein the cartridge having a filter member is a nucleic acid extraction cartridge for extracting nucleic acid, and the specific substance is nucleic acid.

According to this apparatus for recovering a specific substance, the cartridge is a nucleic acid extraction cartridge for extracting nucleic acid, so it is possible to extract nucleic acid in a sample solution.

### BRIEF DESCRIPTION OF THE DRAWING

FIG. 1 is a perspective view showing one embodiment of the nucleic acid extracting apparatus wherein the frontal cover is opened;

FIG. 2 is a perspective exterior view of the nucleic acid extracting apparatus wherein the frontal cover is closed;

FIG. 3 is a schematic constitution diagram of a moving head of the nucleic acid extracting apparatus;

FIG. 4 is a schematic block diagram of the nucleic acid extracting apparatus;

FIG. 5 is a perspective view showing a state in which a cartridge holder, a container holder and a container holding part are imposed integrally as a holding device, and mounted on a mounting part;

FIG. 6 is a perspective view of a cartridge holder, a container holder, a container holding part and a mounting part;

FIG. 7 is a perspective view of the main body of the apparatus wherein the holding device and the liquid container are removed;

FIG. 8 is a perspective view of the main body of the apparatus, in which FIG. 8(a) is a perspective view showing the status where the mounting part is mounted on the main body of the apparatus, and FIG. 8(b) is a perspective view showing the status where the holding device is further mounted on the main body of the apparatus;

FIG. 9 is a schematic block diagram of the main constitution which decides if or not it is a cartridge which is a subject to be treated, for which nucleic acid extraction is carried out;

FIG. 10 shows a flow chart of procedures to decide if or not it is a cartridge which is a subject to be treated;

FIG. 11 is a graph which represents time-dependent pressure change in the first pattern, and a profile in the case that it reaches the peak pressure within the set time;

FIG. 12 is a graph which represents time-dependent pressure change in the second pattern, and a profile in the case that it does not reach the peak pressure within the set time, and further the pressure becomes a maximum within the set time;

FIG. 13 is a graph which represents time-dependent pressure change in the third pattern, and a profile in the case that it does not reach the peak pressure within the set time, and further the pressure does not become a maximum within the set time;

FIG. 14 is a graph which represents time-dependent pressure change in the case that a cartridge is not provided with, and a profile of the pulsation of an air pump;

FIG. 15 is an explanation view showing states (a) to (e) of supplying pressurized air to each of cartridges from a pressurizing nozzle;

FIG. 16(a) is a perspective view of a cartridge and FIG. 16(b) is a XVIB-XVIB section view of a cartridge; and

FIG. 17 shows process views (a) to (g) of the extraction operation,

wherein 4 denotes Pressurized air supply device (Pressurized air supply means); 11 denotes Cartridge (cartridge for nucleic acid extraction); 11b denotes Filter member (Nucleic acid-adsorbing porous membrane); 41 denotes Pressurizing nozzle; 46 denotes Pressure sensor (Pressure detection means); 51w, 51r denote Injection nozzle; 61 denotes Cartridge holder; 70 denotes Control apparatus (means for deciding a subject to be treated); 100 denotes Nucleic acid extracting apparatus (Apparatus for recovering a specific substance); S denotes Sample solution; W denotes Washing solution; and R denotes Recovering solution.

### BEST MODE FOR CARRYING OUT THE INVENTION

Hereinafter, the apparatus for recovering a specific substance and a nucleic acid extracting apparatus using the same of the present invention will be illustrated for preferred embodiments of them, particularly taking examples of the nucleic acid extracting apparatus.

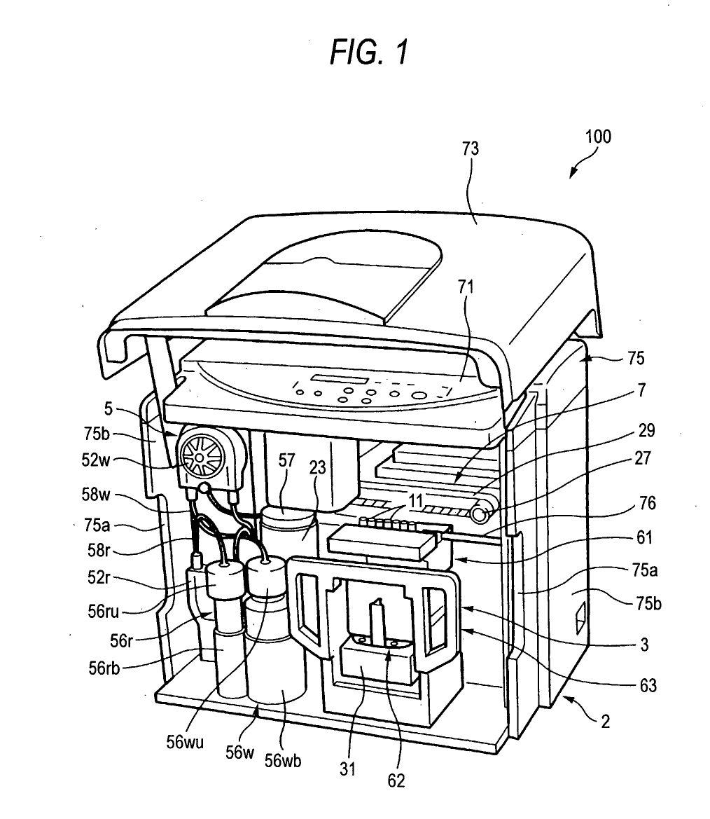

FIG. 1 is a perspective view showing one embodiment of the nucleic acid extracting apparatus wherein the frontal cover is opened.

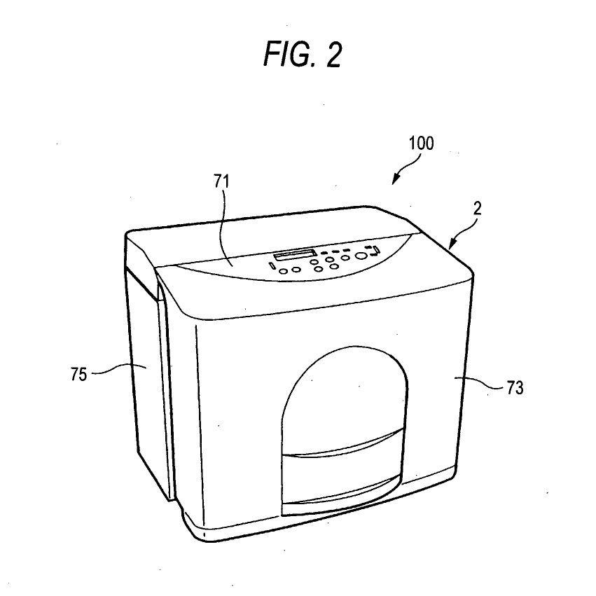

FIG. 2 is a perspective exterior view of the nucleic acid extracting apparatus wherein the frontal cover is closed.

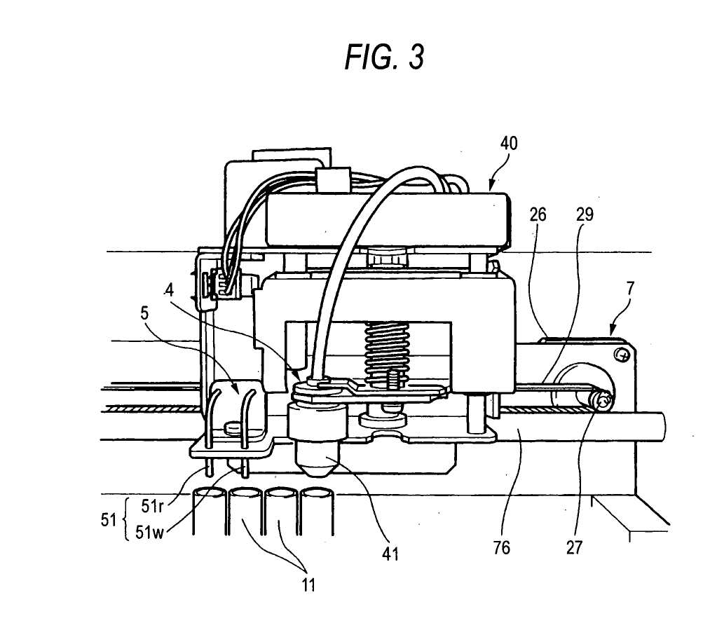

FIG. 3 is a schematic constitution diagram of a moving head of the nucleic acid extracting apparatus.

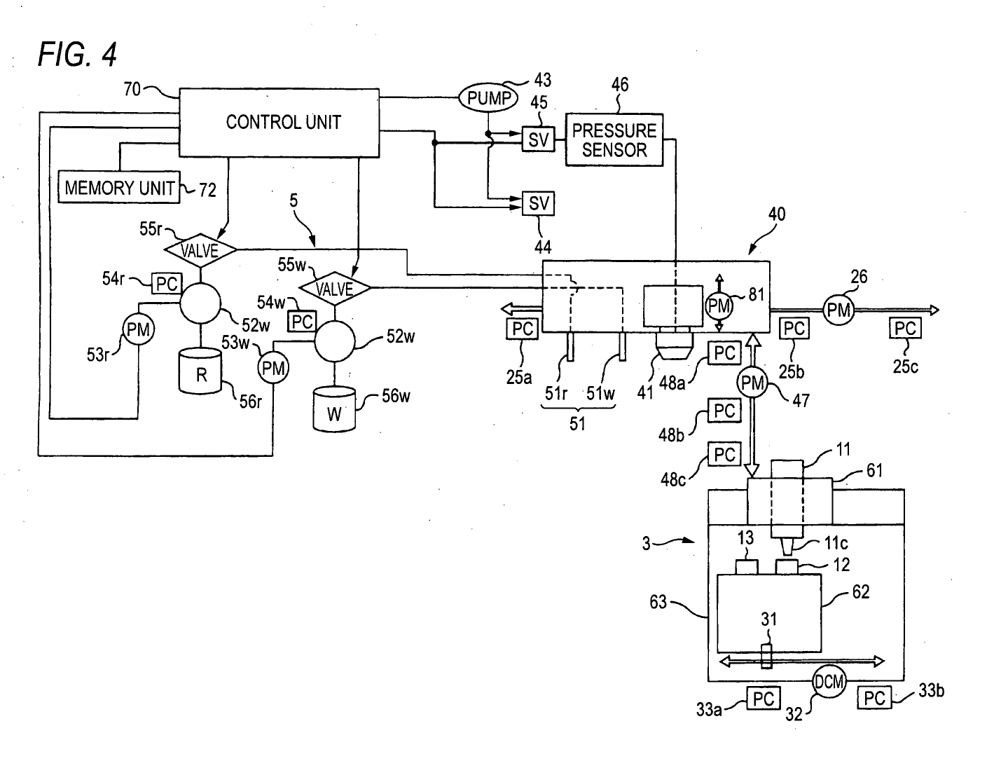

FIG. 4 is a schematic block diagram of the nucleic acid extracting apparatus.

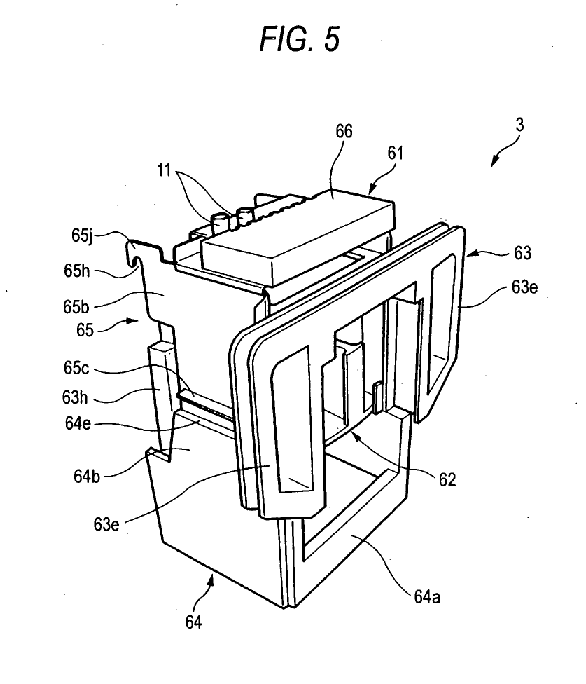

FIG. 5 is a perspective view of a holding device.

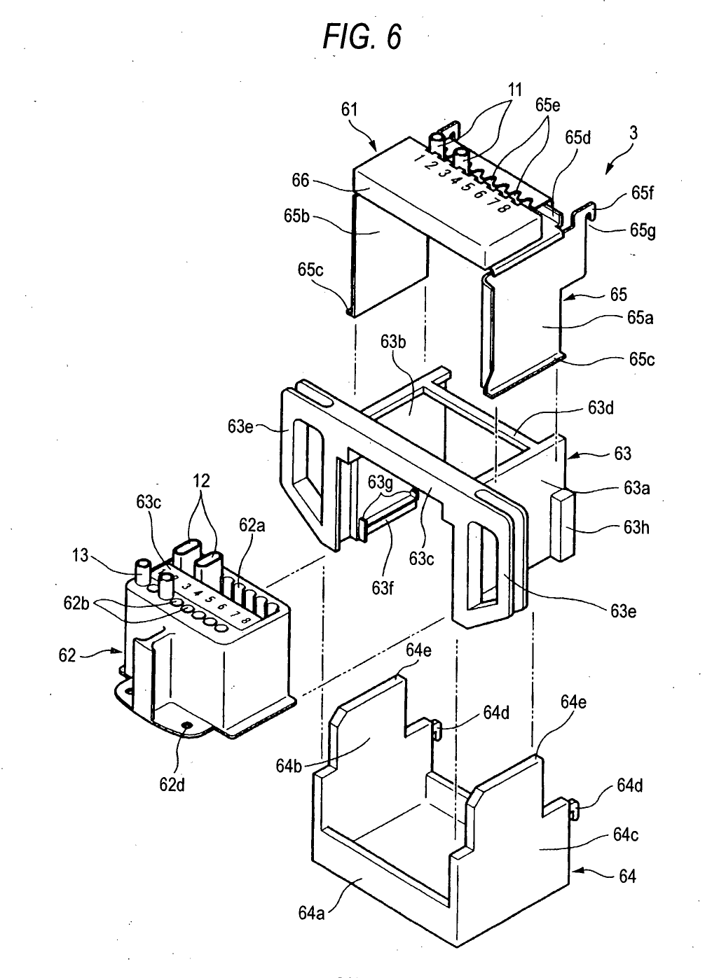

FIG. 6 is a decomposed perspective view of the holding device.

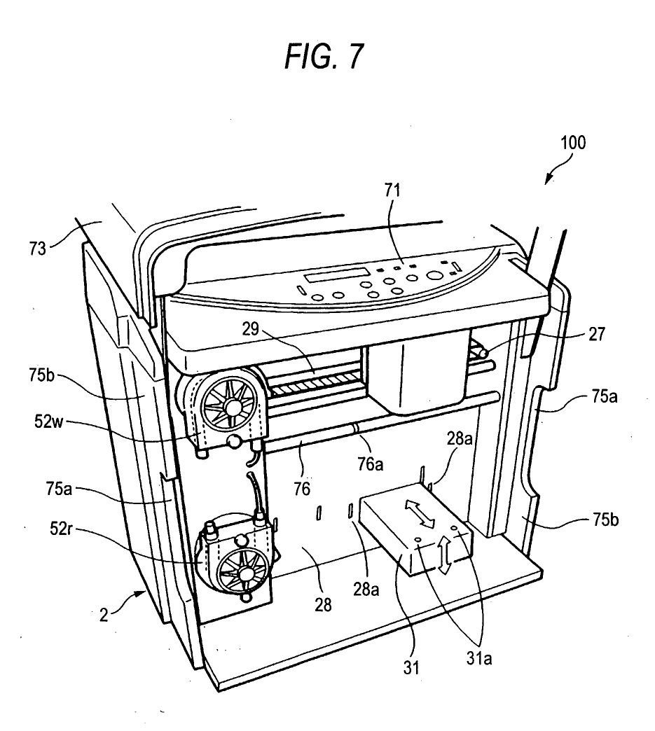

FIG. 7 is a perspective view of the main body of the apparatus wherein the holding device and the liquid container are removed.

The present nucleic acid extracting apparatus 100 is constituted as provided with a holding device 3 which holds a nucleic acid extraction cartridge 11 which receives a filter member in the container (hereinafter, simply referred to as the cartridge), a waste liquor container 12 which receives waste liquor and a recovering container 13 which receives a recovering solution containing nucleic acid, wherein each of them are arranged in plural number, a pressurized air supplying device 4 which introduces pressurized air to the cartridge 11 from a single pressurizing nozzle 41, a separate injection device 5 which has an injection nozzle 51 injecting a washing solution and a recovering solution, respectively to the cartridge 11, and a moving device 7 which moves relatively the pressurizing nozzle 41 of the pressurized air supplying device 4, and the holding device 3. As the filter member, nucleic acid-adsorbing porous solid phase (herein, nucleic acid-adsorbing porous membrane as an example) is used.

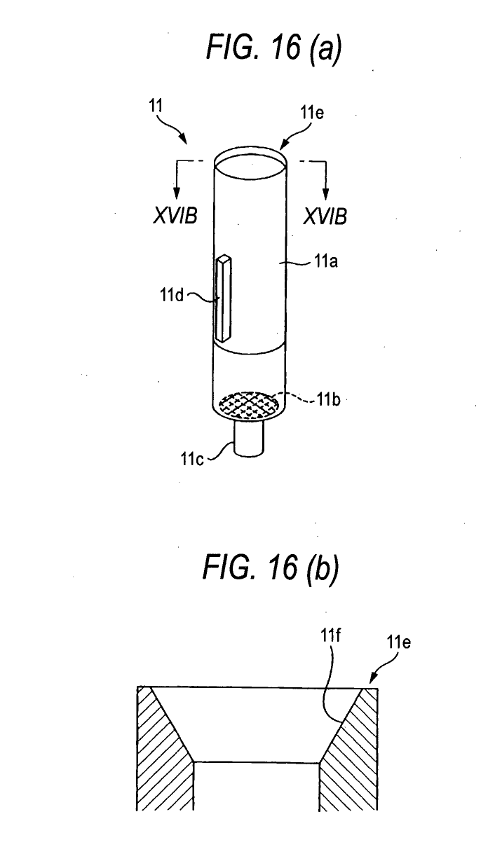

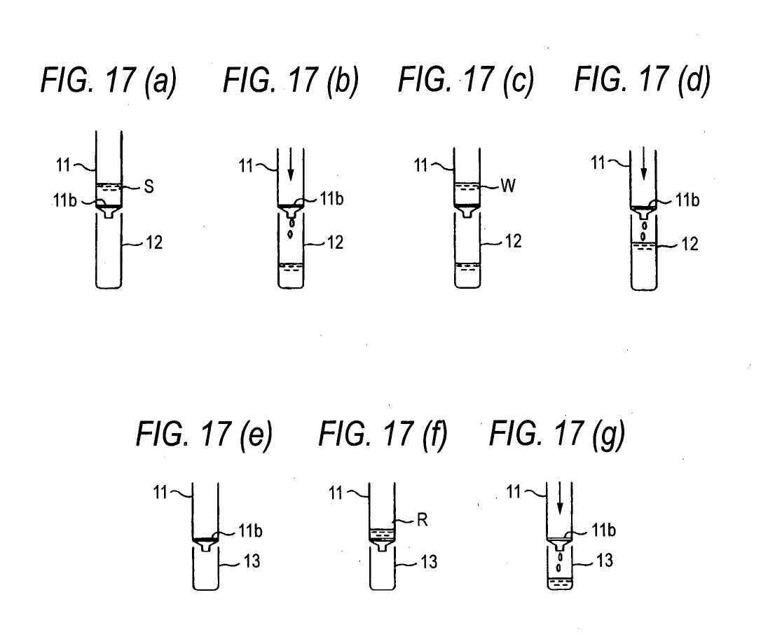

For the cartridge used in the nucleic acid extracting apparatus 100 of the present embodiment, the same cartridge 11 as previously described with reference to FIG. 16 is used.

The nucleic acid extracting apparatus 100 conducts sequentially (1) a process of passing a sample solution containing nucleic acid through nucleic acid-adsorbing porous membrane, to adsorb nucleic acid in the porous membrane, (2) a process of washing the nucleic-adsorbing porous membrane with nucleic acid adsorbed, and (3) a process of passing a recovering solution through nucleic acid-adsorbing porous membrane, to separate nucleic acid from the porous membrane.

In the nucleic acid extracting apparatus 100, the main body of the apparatus 2 is provided with a holding device 3, a pressurized air supplying device 4 which introduces pressurized air to a cartridge 11, a separate injection device 5 which injects a washing solution and a recovering solution to the cartridge 11, etc. as shown in FIGS. 1 to 4.

The main body of the apparatus 2 is provided with a main body part 75 of box shape wherein a control panel 71 is provided on the top side and the frontal side is open, and a frontal cover 73 which covers the open side of the main body part 75, with receiving the holding device 3, the pressurized air supplying device 4, the separate injection device 5 and the moving device 7, etc. On the wall 75b of the lateral side of the front of the main body part 75, a concave part 75a is built which is concave from the frontal side to the back side. According to this, an operation space is reserved on the lateral side of a container holding part 63, which will be described below, and thereby, when detaching the container holding part 63 to the main body of the apparatus 2, it prevents the interference by a hand grasping the container holding part 63 and etc. to the main body part 75, which leads to improvement of workability.

Next, the holding device 3, the pressurized air supplying device 4 and the separate injection device 5 will be explained specifically.

### <Holding Device>

The holding device 3 comprises a cartridge holder 61, a container holder 62 and a container holding part 63 as shown in FIGS. 5 and 6. In the container holding part 63, the cartridge holder 61 and a container holder 62 are placed as set in location. The container holding part 63 wherein the cartridge holder 61 and the container holder 62 are placed, is further placed on a mounting part 64.

As shown in FIG. 7, the movement for exchanging containers of the container holder 62 (forward and backward movements), is carried out by movement of an operation member 31, which is projected to the front from the rear wall 28 of the main body of the apparatus 2 and installed movably in back and forth direction and up and down direction, in response to the drive of a container exchange motor 32 (DC motor). By the back and forth movement, a recovering container 13, or a waste liquor container 12 is located below the cartridge 11 which is held in the cartridge holder 61. The operation of the container exchange motor 32 is controlled according to the detection of location sensors 33a and 33b.

In the mounting part 64, both of lateral walls 64b and 64c are installed as projected upward from a base unit 64a which is formed in substantially rectangular frame shape. On the upper rear of the both of lateral walls 64b and 64c, a hook unit 64d of substantially reverse U letter shape is formed as projected to the rear.

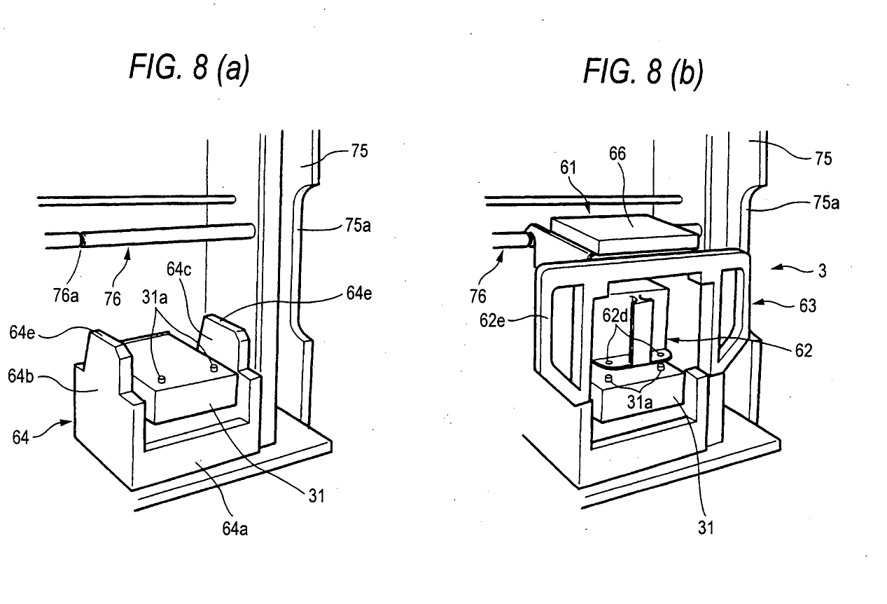

Herein, FIG. 8 is a perspective view of the main body of the apparatus, in which FIG. 8(a) is a perspective view showing the status where the mounting part is mounted on the main body of the apparatus and FIG. 8(b) is a perspective view showing the status where the holding device is further mounted on the main body of the apparatus.

As shown in FIG. 8(a), the mounting part 64 is inserted and engaged into a rectangular locking hole 28a (see FIG. 7), in which the hook unit 64d is formed on a rear wall 28 of the main body of the apparatus 2, and thereby, a base unit 64a is located below an operation member 31, and at the same time, the both of lateral walls 64b and 64c are placed on both of the lateral sides of the operation member 31, to be installed in the main body of the apparatus 2. Accordingly, the operation member 31 is movable between the both of lateral walls 64b and 64c in the back and forth direction and the up and down direction.

As shown in FIG. 8(b), the holding device 3 wherein the cartridge holder 61, the container holder 62 and the container holding part 63 are combined integrally, is placed on the mounting part 64 which is installed in the main body of the apparatus 2.

The cartridge holder 61 is provided with a holder 65, which is formed by bending a stainless steel plate, etc. into an approximately “U” shape, and a plate material 66, and is constructed in a two-divided structure. The bottoms of both of lateral walls 65a and 65b of the holder 65 are bent in a direction to apart from each other, to form a supporting part 65c. In addition, on the upper back ends of both of the lateral walls 65a and 65b, locking parts 65f and 65j are formed having locking grooves 65g and 65h of an approximately reverse “U” shape (See FIG. 5 and FIG. 6). These locking parts 65f and 65j are engaged with a locking rod 76 and a notch groove of the locking rod 76 to be positioned in alignment with each other.

The back end of a middle part 65d which connects both of the lateral walls 65a and 65b is further bent into an approximately “U” shape, and at the same time, is provided with plural V-shaped holding grooves 65e, which are formed in a V shape (8 places in the embodiment shown in the drawing).

The plate material 66 is constructed to be movable in a direction for connecting and separating to/from the V-shaped holding grooves 65e of the holder 65, and biased in a direction adjacent to the V-shaped holding grooves 65e by a coil spring (not shown) contained therein. In addition, on the plate material 66, plural V-shaped holding parts (not shown) are formed at a location corresponding to the V-shaped holding grooves 65e of the holder 65, and between the V-shaped holding grooves 65e of the holder 65 and the plate material 66, a cartridge 11 is sandwiched by the elastic force of the coil spring. In other words, a grasping device of the cartridge 11 is constructed by the V-shaped holding grooves 65e of the holder 65, the holding part of the plate material 66, and the coil spring.

For the cartridge 11 sandwiched by the grasping device, projections 11d (see FIG. 16), which are formed on both sides of the lateral part of a cylindrical main body 11a, are engaged and held with an engagement part (not shown) of the plate material 66. If the plate material 66 is moved against the elastic force of the coil spring, the engagement with the projections 11d are released to drop and discard all the cartridges 11 at the same time downward. In addition, numbers are written in ascending order at the locations corresponding to each of the holding parts of the plate material 66, which makes it possible to identify easily the held cartridges 11 individually.

In the container holding part 63, one pair of lateral walls 63a and 63b, which are connected by ribs 63c and 63d, are arranged in the opposite direction, as shown in FIG. 6. On the rib 63c, one pair of grasping members 63e are further formed to be extended and installed on both of the lateral sides. In addition, on the lower part of the inner wall surface of the one pair of the lateral walls 63a and 63b, one pair of supporting ribs 63f, which are opposite to each other, are formed in the horizontal direction, and on the supporting ribs 63f, a container holder 62 can be mounted. On the both ends of the upper side of the supporting ribs 63f, projections 63g, which projects upward, are formed, and a container holder 62, which is mounted on the supporting ribs 63f, abuts on the projections 63g to be arranged in a positioning state in the back and forth direction. Furthermore, on the front of the outer wall surfaces of the one pair of the lateral walls 63a and 63b, a longitudinal rib 63h is formed in the up and down direction. A cartridge holder 61 is inserted from the upper side between the longitudinal rib 63h and the grasping member 63e, with the one pair of the lateral walls 63a and 63b sandwiched between both of the lateral walls 65a and 65b, and mounted into the container holding part 63 in a positioned state.

The container holder 62 is provided with waste liquor container holding holes 62a and recovering container holding holes 62b in parallel two rows, which extend on the inner top surface in the across direction. Plural waste liquor containers 12 are held at the waste liquor container holding holes 62a on the rear side, and plural recovering containers 13 are held in the recovering container holding holes 62b on the front side, respectively in a row shape. The waste liquor container holding holes 62a and the recovering container holding holes 62b are placed and installed at the equal location with the equal pitch to that of the grasping device of the cartridge holder 61 (V-shaped holding groove 65e), and waste liquor containers 12 and recovering containers 13 are set to be located below each of the held cartridges 11.

On the inner top surface 62c between the waste liquor container holding holes 62a and the recovering container holding holes 62b, which were formed in two rows, numbers corresponding to those written in the cartridge holder 61 are written in ascending order. According to this, it is possible to identify the cartridge 11 held in the cartridge holder 61, and the waste liquor containers 12 and the recovering containers 13 held in the container holder 62 as matched one by one. In addition, on the bottom side of the container holder 62, one pair of positioning holes 62d are formed. Furthermore, to prevent confusion of the waste liquor containers 12 with the recovering containers 13, these are preferably different in size, shape, etc.

As shown in FIG. 5, in the cartridge holder 61, both of the lateral walls 65a and 65b are placed as inserted from the upper side of the container holding part 63 such that one pair of the lateral walls 63a and 63b are sandwiched therebetween. In addition, the container holder 62 is inserted from an opening on the front side of the container holding part 63 to be mounted on one pair of supporting ribs 63f. According to this, the cartridge holder 61, the container holder 62 and the container holding part 63 are integrally assembled to constitute the holding device 3. The holding device 3 is mounted on the mounting part 64 which is placed on the main body of the apparatus 2, and at this time, a supporting part 65c of the cartridge holder 61 is held abutting on the both of the lateral walls 64b and 65c of the mounting part 64.

At a lowered position where the container holder 62 is mounted on the pair of the supporting ribs 63f (see FIG. 6) of the container holding part 63, as shown in FIG. 5, the lower end of a discharge part 11c of the cartridge 11 held in the cartridge holder 61 (see FIG. 16), is located on the upper side than the waste liquor container 12 and the recovering container 13, which are set in the container holder 62. If the container holder 62 is operated up and down by the drive of a shifting motor 47 such as a pulse motor (see FIG. 4), and thereby the container holder 62 is moved up and down by a control involved in the detection of photo sensors 48a to 48c, and according to this, the discharge part 11c of the cartridge 11 is inserted in a predetermined amount into the waste liquor container 12 and the recovering container 13 when the container holder 62 is elevated.

<Pressurized Air Supply Device>

The pressurized air supply device 4 is provided as shown in FIG. 4 with a moving head 40 as a movable body moving up and down for the container holder 62, a single pressurizing nozzle 41 which is installed in this moving head 40, an air pump 43 which generates pressurized air, a relief valve 44, a check valve 45 which opens and closes air supply pathway installed on the side of the pressurizing nozzle 41, a pressure sensor 46 which is installed on the side of the pressurizing nozzle 41, and a nozzle shifting means which lifts or lowers the pressurizing nozzle 41. The nozzle shifting means achieves the lifting and lowering operation by a nozzle shifting motor 81 such as a pulse motor and a screw nut device which is connected to this. According to this construction, pressurized air is supplied to the cartridge 11 in order. The air pump 43, the relief valve 44 and the pressurizing nozzle 41 are operated, respectively on the base of the control instruction from a control unit 70.

The moving head 40 is provided with a head moving motor 26 such as a pulse motor as a moving means which is installed in the inner part of the main body of the apparatus 2 (see FIG. 3 and FIG. 4), a driving-side pulley 27 which is driven to rotate by the head moving motor 26, a vertically moving-side pulley which is rotatable and conducts tension adjustment (not shown), and a timing belt 29 which is suspended between the driving-side pulley 27 and the vertically moving-side pulley. Furthermore, the head moving motor 26 is driven by a control involved in the detection of photo sensors 25a to 25c, to move the moving head 40 along the arrangement direction of the cartridges 11.

The pressurizing nozzle 41 is installed as movable up and down and biased below the moving head 40, and the outer circumferential side of the lower tip of the pressurizing nozzle 41 is in the conic shape. According to this, when the pressurizing nozzle 41 is moved downward, the tip of the pressurizing nozzle 41 abuts on the upper opening 11e of the cartridge 11 which is set in the cartridge holder 61, and thereby, the inclined surface 11f, which is cut as a taper shape of the cartridge 11, is closely attached to the conic side of the tip of the pressurizing nozzle 41 to seal the inside of the cartridge 11. Under such a sealed state, it is possible to supply pressurized air into the inside of the cartridge 11 without leakage.

The relief valve 44 is operated to be open to the air when discharging the air in the pathway between the air pump 43 and the check valve 45. The check valve 45 is selectively operated to be opened, to constitute an air circuit so that pressurized air from the air pump 43 is introduced into the inside of the cartridge 11 through the pressurizing nozzle 41. According to the construction as described above, a flow way to supply the air is formed from the air pump 43 to the cartridge 11.

<Separate Injection Device>

The Separate injection device is provided as shown in FIGS. 1, 3, 4 and 7 with a washing solution separate injection nozzle 51w and a recovering solution separate injection nozzle 51r, which are mounted integrally on the above-mentioned moving head 40 that is movable on the cartridge holder 61 in the parallel direction of the cartridge 11, a washing solution feeding pump 52w which supplies a washing solution W, that is received in a washing solution bottle 56w, to the washing solution separate injection nozzle 51w, a recovering solution feeding pump 52r which supplies a recovering solution R, that is received in a recovering solution bottle 56r, to the recovering solution separate injection nozzle 51r, and a waste liquor container 57 which is placed in a waste liquor container die 23, etc.

The moving head 40 stops on each of the cartridges 11 in order by the head moving motor 26 (see FIG. 4), and stops on the waste liquor container 57 in the returning state, to be driven and controlled to empty the space on each of the cartridges 11. By emptying the space on each of the cartridges 11, workability is largely improved.

The washing solution separate injection nozzle 51w and the recovering solution separate injection nozzle 51r are curved with the tip downward. The washing solution separate injection nozzle 51w is connected to the washing solution feeding pump 52w via the valve 55w, and the washing solution feeding pump 52w is connected to the washing solution supplying bottle 56w. The recovering solution separate injection nozzle 51r is connected to the recovering solution feeding pump 52r via the valve 55w, and the recovering solution feeding pump 52r is connected to the recovering solution supplying bottle 56r. The washing solution bottle 56w and the recovering solution bottle 56r are provided, respectively on the frontal side of the main body of the apparatus 2 to enhance operability. The washing solution feeding pump 52w and the recovering solution feeding pump 52r are constituted as a tube pump, and driven and controlled to inject a washing solution W and a recovering solution R in a prescribed amount by pump motors 53w and 53r (pulse motors), respectively on the base of the location detection of sensors 54w and 54r. These pump motors 53w and 53r, and valves 55w and 55r are operated on the base of the instruction from the control unit 70.

When the washing solution W or the recovering solution R are injected, the valves 55w and 55r are opened, and the pump motors 53w and 53r are driven to operate rotation of a rotor member of the washing solution feeding pump 52w or the recovering solution feeding pump 52r. According to this, the washing solution W or the recovering solution R are aspirated by the washing solution feeding pump 52w or the recovering solution feeding pump 52r to be discharged from the washing solution separate injection nozzle 51w or the recovering solution separate injection nozzle 51r through the valves 55w or 55r. At this discharge, the washing solution separate injection nozzle 51w or the recovering solution separate injection nozzle 51r is placed as moved on the cartridge 11. According to this, the washing solution W or the recovering solution R are injected to the cartridge 11 in a prescribed amount.

The washing solution bottle 56w and the recovering solution bottle 56r comprise container main bodies 56wb and 56rb, and caps 56wu and 56ru. On both of the caps 56wu and 56ru, suction tubes 58w and 58r of the fine pipe shape are installed, respectively, and the lower end of the suction tubes 58w and 58r are open near the bottom of the container main bodies 56wb and 56rb as to aspirate the washing solution W and the recovering solution R in response to the operation of the washing solution feeding pump 52w or the recovering solution feeding pump 52r.

Each of the devices 3 to 5 as described above, is controlled by a control unit 70 (see FIG. 4) linked, in correspondence to the input manipulation at a manipulation panel 71, which is installed on the upper part of main body of the apparatus 2. In conclusion, it is driven and controlled on the base of the program that is memorized in advance on a memory unit 72, which is connected to the control unit 70. In addition, as shown in FIG. 1 and FIG. 2, each of the devices 3 to 5 is received in the main body of the apparatus 2 by covering the front of the main body of the apparatus 2 with a frontal cover 73, which is arranged to be able to open and close.

Next, extraction operation by the nucleic acid extracting apparatus 100 of the above construction will be illustrated in detail.

First, decision of a cartridge which is a subject to be treated, for which recovery of a specific substance (herein, nucleic acid) is conducted, will be illustrated, which constitutes the main part of the present invention.

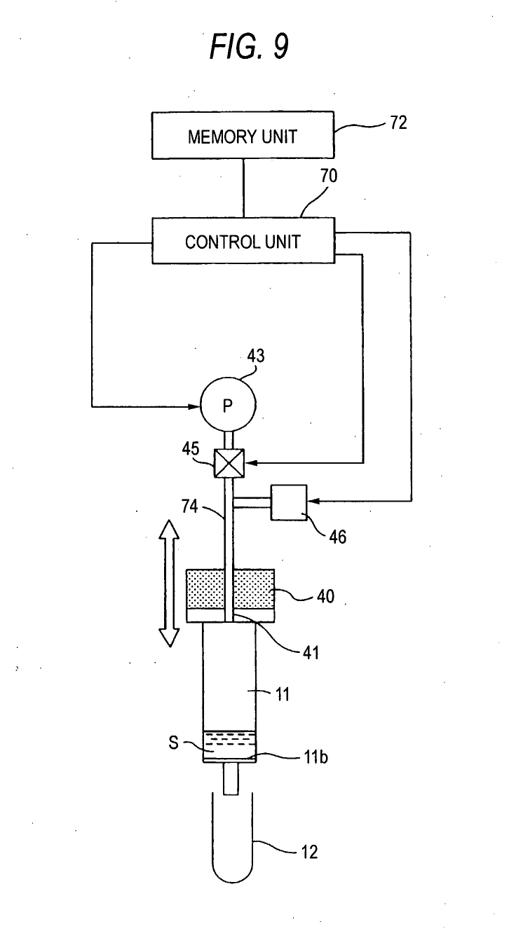

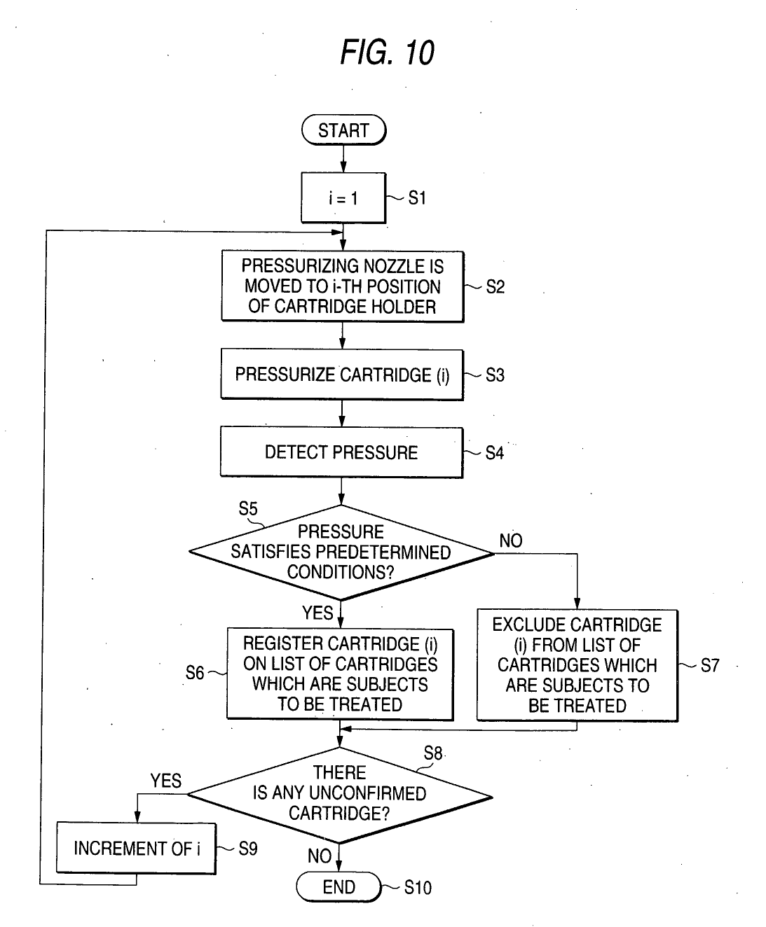

FIG. 9 is a schematic block diagram of the main construction which decides if or not it is a cartridge which is a subject to be treated, for which nucleic acid extraction is carried out. FIG. 10 shows a flow chart of procedures to decide if or not it is a cartridge which is a subject to be treated.

As shown in FIG. 9, a decision device which decides if or not it is a cartridge which is a subject to be treated, for which nucleic acid extraction is carried out, is provided with a moving head 40 that moves up and down for the cartridge 11. The moving head 40 is connected to an air pump 43 via an electromagnetic valve 45. In addition, on the route of a pipe 74 which connects the pressurizing nozzle 41 and the electromagnetic valve 45, a pressure sensor 46 is mounted to measure the pressure in the pipe 74, and the measurement results are input to the control unit 70. On the base of the data of the measured pressure in the pipe 74, the control unit 70 decides if or not it is a cartridge which is a subject to be treated, for which nucleic acid recovery is carried out, according to the program that is memorized on the memory unit 72. In other words, the control unit 70 works as a means for deciding a subject to be treated which decides if or not it is a cartridge which is a subject to be treated, for which nucleic acid (a specific substance) extraction is conducted, by the pressure detected by the pressure sensor 46 when the pressurized air is introduced into the cartridge 11.

The pressure signal being input to the control unit 70 is sampled in a prescribed time interval, and the average value per one second is always calculated to avoid mis-decision of algorithm by noise. The prescribed time interval is preferably less than 0.5 second.

Next, an algorithm will be illustrated, which decides if or not it is a cartridge which is a subject to be treated, for which nucleic acid extraction should be conducted.

As shown in the flow chart of FIG. 10, a memory value of a counter i is set to 1 (Step 1, hereinafter referred simply to as S1), then the pressurizing nozzle 41 is moved to closely attach to the first cartridge (C1) (S2), and pressurized air from the air pump 43 is supplied to the cartridge (C1) (S3) to measure the pressure in the pipe 74 (S4). Then, it is discriminated if the measured pressure satisfies a prescribed condition (S5). If the measured pressure satisfies a prescribed condition, the cartridge (C1) is registered the list of the cartridges which are subjects to be treated (S6), and if not, it is excluded from the list of the list of the cartridges which are subjects to be treated. Next, presence or absence of a cartridge for which the above decision has not been conducted yet (S8), is discriminated, and if it is a cartridge for which the above decision has not been conducted yet, the memory value of the counter i is subjected to increment (S9), to return before Step 2 and conduct the same manipulations again in repetition. By doing this, if decision for all the cartridge is completed, the decision is completed if or not it is a cartridge which is a subject to be treated (S10).

To decide that it is a cartridge which is a subject to be treated, the prescribed condition that the measured pressure should satisfy, is largely different depending on the kinds of the filter member and the sample solution, so it is classified herein into three typical patterns.

(First Pattern)

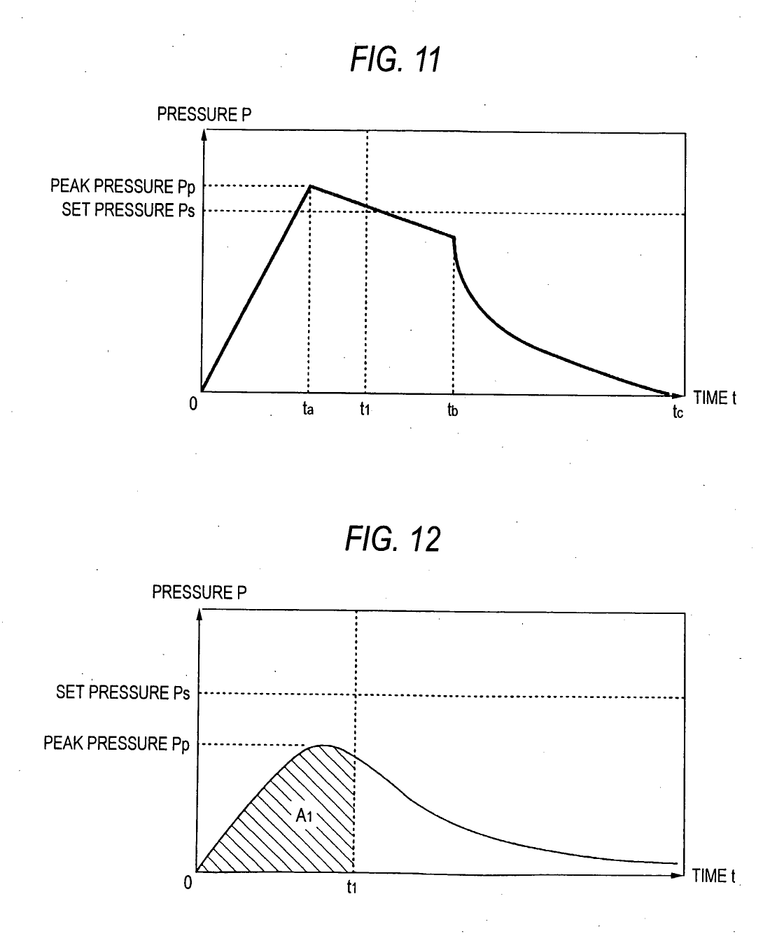

FIG. 11 is a graph which represents time-dependent pressure change in the first pattern, and a profile in the case that it reaches the peak pressure within the set time. In this profile, if pressurized air is supplied to the cartridge 11 by the pressurizing nozzle 41, the pressure in the cartridge 11 increases to show a peak value Pa at a time ta. In other words, it reaches the peak value Pa, which is more than the prescribed set value Ps within a prescribed time t1, which is set in advance. Then, a sample solution S in the cartridge 11 passes a nucleic acid-adsorbing porous membrane (a filter member) 11b to be discharged to the outside of the cartridge 11, and according to this, the pressure decreases gradually, and if all the sample solution S is discharged, the pressure in the cartridge 11 is rapidly reduced.

For such cartridge 11 which shows the pressure profile of the first pattern, the cartridge 11 which has been a subject is decided as a cartridge which is a subject to be treated by reaching the peak value Pa, which is more than the prescribed set value Ps within a prescribed time t1, which is set in advance.

(Second Pattern)

FIG. 12 is a graph which represents time-dependent pressure change in the second pattern, and a profile in the case that it does not reach the peak pressure within the set time, and further the pressure becomes a maximum within the set time. In this profile, if pressurized air is supplied to the cartridge 11 by the pressurizing nozzle 41, the pressure in the cartridge 11 increases, but the increasing rate of the pressure decreases as time passes and it shows a peak value Pp and then it decreases for a short time. The peak value Pp of the pressure is lower than the prescribed set value Ps, and it does not reach the prescribed set value Ps within a prescribed time t1, which is set in advance.

For such cartridge 11 which shows the pressure profile of the second pattern, the cartridge 11 which has been a subject is decided as a cartridge which is a subject to be treated when an integral value A1 of the pressure in the cartridge 11 within a prescribed time t1, is more than a value which is set in advance.

(Third Pattern)

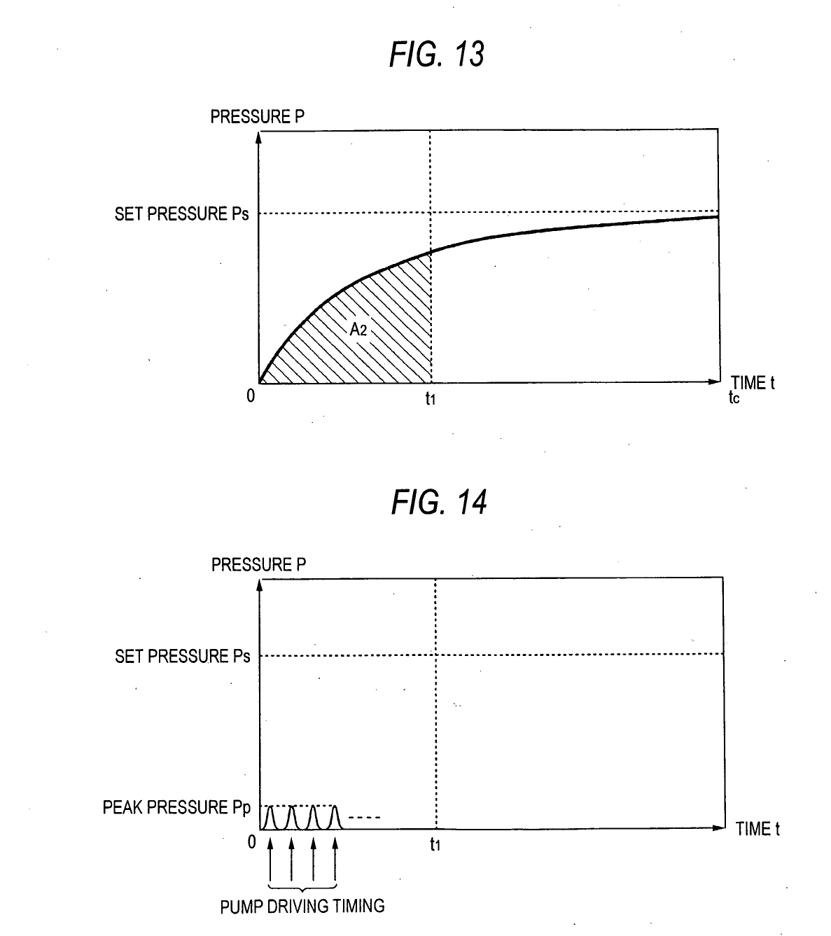

FIG. 13 is a graph which represents time-dependent pressure change in the third pattern, and a profile in the case that it does not reach the peak pressure within the set time, and further the pressure does not become a maximum within the set time. In this profile, if pressurized air is supplied to the cartridge 11 by the pressurizing nozzle 41, the pressure in the cartridge 11 increases, but the increasing rate of the pressure decreases as time passes and it shows no peak value.

For such cartridge 11 which shows the pressure profile of the third pattern, the cartridge 11 which has been a subject is decided as a cartridge which is a subject to be treated when an integral value A2 of the pressure in the cartridge 11 within a prescribed time t1, is more than a value which is set in advance, similarly to the cartridge 11 showing the pressure profile of the second pattern.

(Pattern of Irregular Pressure)

FIG. 14 is a graph which represents time-dependent pressure change in the case that a cartridge is not provided with, and a profile of the pulsation of an air pump. In this profile, since a sample solution S is not injected into the cartridge 11, the pressure measured by the pressure sensor 46 shows no increase, and only the pulsation of the air pump 43 is detected as shown in the figure.

When the pressure profile of such irregular pressure pattern is obtained, it is decided that it is not a cartridge which is a subject to be treated.

Next, extraction operation by the nucleic acid extracting apparatus 100 will be illustrated in detail.

First, as shown in FIG. 8(a), a hook unit 64d of the mounting part 64 is inserted and engaged to a rectangular locking hole 28a (see FIG. 7), which is formed on the rear wall 28 of the main body of the apparatus 2, to locate a base unit 64a below an operation member 31, and further both of the lateral walls 64b and 64c are installed in the main body of the apparatus 2 that both sides of the operation member 31 are sandwiched between them.

Next, a cartridge 11 is set in the cartridge holder 61 of the holding device 3, which is taken out to the outside of the main body of the apparatus 2, and placed in the container holding part 63, and further a container holder 62 holding a waste liquor container 12 and a recovering container 13 is placed on one pair of supporting ribs 63f of the container holding part 63. Then, a sample solution S which is treated as dissolved, is injected to each of the cartridges 11 in order with a pipet, etc.

For the preparation tasks as described above, the cartridge 11, the waste liquor container 12 and the recovering container 13 are not needed to be set for all of the holding units of the cartridge holder 61 and the container holder 62, but are set in any number corresponding to that of the sample solution S to be extracted for nucleic acid. In addition, the location of the cartridge 11, the waste liquor container 12 and the recovering container 13 to be set are optional for the waste liquor container 12 and the recovering container 13, and the waste liquor container 12 and the recovering container 13 may be set at a location corresponding to the location of the cartridge 11.

For the holding device 3 which is imposed as described above, a grasping member 63e of the holding device 3 is grasped by a worker and placed in a mounting part 64 which is installed in the main body of the apparatus 2, as shown in FIG. 8(b). At this time, on a wall 75b of the frontal lateral side of the main body part 75, a concave part 75a is built which is concave from the frontal side to the back side, so an operation space is reserved, and therefore, when detaching the container holding part 63 from the main body of the apparatus 2, it is possible to work easily without interference to the main body part 75 by a hand grasping the container holding part 63, and etc.

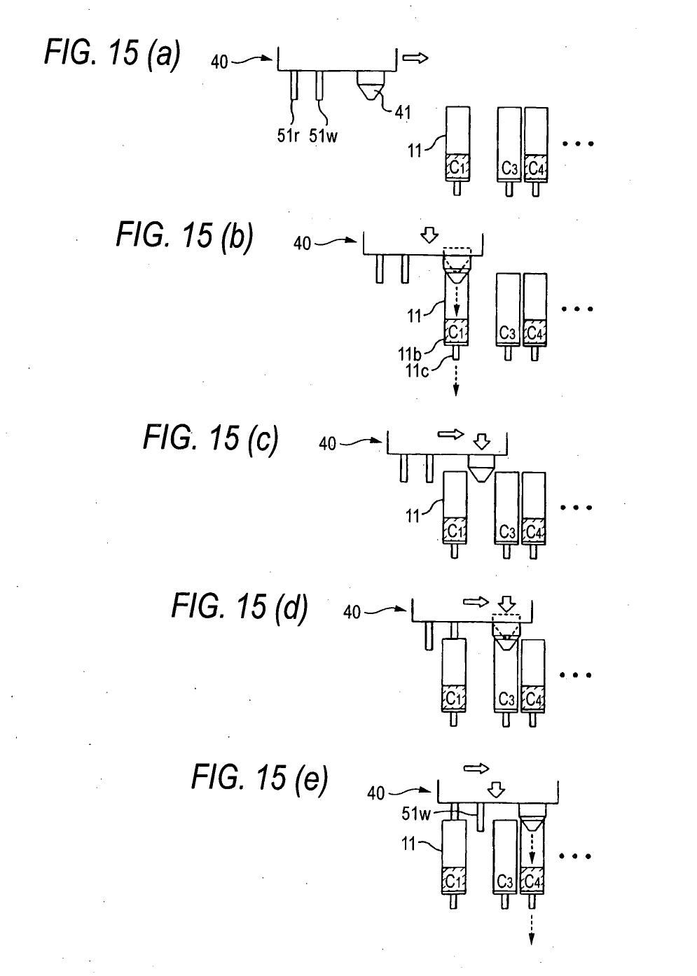

Here, FIGS. 15 (a) to (e) are illustrative diagrams of supplying pressurized air to each cartridge from the pressurizing nozzle. Hereinafter, it will be described with appropriate reference to FIG. 15.

Thereafter, if the apparatus is operated by controlling the control panel 71, a moving head 40 is driven to the location immediately above of the cartridge 11. Then, as an example, the pressurizing nozzle 41 is arranged to the immediate above of the cartridge (C1) at the left end in the figure, and the pressurizing nozzle 41 of the moving head is driven downward by driving a nozzle shifting motor 81 of the pressurized air supply device 4 that the outer peripheral surface at the tip of the pressurizing nozzle 41 attaches to the inclined surface 11f of the cartridge (C1) (FIG. 15(b)).

Meanwhile, as shown in FIG. 8(b) which shows the location relationship between the container holder and the operation member, if the operation member 31 is elevated by driving the shifting motor 47, a pair of positioning pins 31a fit to a pair of positioning holes 62d on the container holder 62 to determine the relative position of the operation member 31 and the container holder 62. Then, further the container holder 62 is elevated to insert the discharge unit 11c of the lower end of the cartridge 11 in predetermined amount into the waste liquor container 12, which prevents that the discharged solution is leaked to the outside by flying, and the like which is one the causes for contamination.

Then, supply of pressurized air is carried out. The air pump 43 is driven with the check valve 45 closed by the command of the control unit 70, and the check valve 45 is operated to be open. Then, pressurized air from the air pump 43 is supplied in a predetermined amount to the first cartridge (C1) through the pressurizing nozzle 41.

Then, the pressure in the cartridge (C1) is measured by a pressure sensor 46, and the measurement results, according to the flowchart in FIG. 10, decide whether the cartridge (C1) is subject to be treated for carrying out the recovery of nucleic acid. For example, if the cartridge (C1) shows the first profile pattern as shown in FIG. 13 wherein it reaches a predetermined pressure Ps within a predetermined time t1, the cartridge (C1) is registered in the list of subjects to be treated in the memory unit 72 as a cartridge which is a subject to be treated.

Next, as shown in FIG. 15(c), following that the check valve 45 is operated to be open, the pressurizing nozzle 41 is elevated by the nozzle shifting motor 81 to drive the head moving motor 26, and thereby moving the moving head 40 in a distance equal to the arrangement pitch of the cartridge 11. Then, with respect to the next second cartridge (C2), pressurized air is supplied in a predetermined amount in the same manner. As for the example shown in the figure, the cartridge (C2) is not provided, thus the pulsation of the air pump 43 is detected as shown in FIG. 14, without increase in the pressure measured by a pressure sensor 46. The peak pressure of the pulsation is far less than the set pressure Ps, and does not satisfy any condition of the first, second and third patterns as described above, thus the cartridge (C2) is excluded from the list of subjects to be treated.

Further, the moving head 40 is moved in a distance equal to the arrangement pitch of the cartridge 11, and for the third cartridge (C3), pressurized air is supplied in a predetermined amount in the same manner (FIG. 15(d)). As for the example shown in the figure, a sample solution S is not injected to the cartridge (C3), thus only the pulsation of the air pump 43 is detected as shown in FIG. 14, without increase of the pressure which is measured by the pressure sensor 46. Accordingly, the cartridge (C3) is excluded from the list of subjects to be treated.

In the same manner, the moving head 40 is moved in a distance equal to the arrangement pitch of the cartridge 11, and for the fourth cartridge (C4), pressurized air is supplied in a predetermined amount in the same manner (FIG. 15(e)). For example, if the measured pressure of the cartridge (C4) shows the profile of the second or third pattern shown in FIG. 12 or FIG. 13 wherein it does not reach the predetermined pressure Ps within the predetermined time t1, but the integral value within the predetermined time t1 is more than the value which is set in the advance, the cartridge (C4) is registered in the list of subjects to be treated in the memory unit 72 as a cartridge which is a subject to be treated.

Then, in the same manner, the supply of the pressurized air, and decision if or not it is a cartridge which is a subject to be treated, are carried out repeatedly in order for all of the cartridges 11 which are held in the cartridge holder 61, and a cartridge 11 which satisfies the condition is registered in the list of subjects to be treated as a cartridge which is a subject to be treated.

For the sample solution S on which the pressure has worked, nucleic acid is held to be adsorbed through the nucleic acid-adsorbing porous membrane 11b, and the other liquid components are discharged to the waste liquor container 12 from the discharge unit 11c of the lower part. When all the sample solution S are passed though the nucleic acid-adsorbing porous membrane 11b, the pressure is reduced to less than the liquid discharge completion pressure, and the completion of desorption of the cartridge 11 is detected by the pressure sensor 46.

Next, the process proceeds to a washing treatment. After the supply of the pressurized air, the moving head 40 is elevated and returned to the position above the initial cartridge (C1). Since the cartridge (C1) is being registered on the list of subjects to be treated as a cartridge which is a subject to be treated, a separate injection nozzle for washing solution 51w of the moving head 40 stops on the first cartridge (C1) to inject the washing solution W in a predetermined amount. Next, when the moving head 40 is moved to the next cartridge, the second cartridge (C2) and the third cartridge (C3) are not registered in the list of subjects to be treated as a cartridge which is a subject to be treated, thus they are skipped and moved to the fourth cartridge (C4) to inject the washing solution W.

Then, in the same manner, only with respect to the cartridges 11 which are registered in the list of subjects to be treated as a cartridge which is a subject to be treated, the washing solution W is injected, and the cartridges 11 which are not registered in the list of subjects to be treated as a cartridge which is a subject to be treated, are skipped. By doing so, when the injection of the washing solution W is completed as for all of the cartridges 11, the moving head 40 is returned to the first cartridge (C1).

Next, the pressurizing nozzle 41 of the moving head 40 is lowered, and the lower part of the pressurizing nozzle 41 is compressed to the upper opening 11e of the cartridge (C1), and sealed, so the check valve 45 is operated to be opened to supply pressurized air to the cartridge (C1) in the same manner as described above. The washing solution W to which the pressure is given, is passed through the nucleic acid-adsorbing porous membrane 11b to perform washing and removing of impurities other than nucleic acid, and the washing solution W is discharged into the washing container 12 from the discharge unit 11c of the lower part.

Also, in this washing process, only for the cartridges 11 which are registered in the list of subjects to be treated as a cartridge which is a subject to be treated, the pressurized air is supplied, and the cartridges 11 which are not registered in the list of subjects to be treated as a cartridge which is a subject to be treated, are skipped. When all the washing solution W is passed through the nucleic acid-adsorbing porous membrane 11b and discharged from the cartridges 11 (cartridges which are registered in the list of subjects to be treated), the moving head 40 is moved to the initial position. Furthermore, in the case of carrying out the washing treatment in multiple times, the above procedures are repeated.

Next, the process proceeds to a recovering treatment. First, at the same time with the moving head 40 returning operation after the washing treatment, the container holder 62 is moved downward by the shifting motor 47, and the lower discharge unit 11c of the cartridge 11 is taken out from the waste liquor container 12, and then the operation member 31 is moved by driving a container exchange motor 32 to move the container holder backward. By doing so, a recovering container 13 is positioned below the cartridge 11 so as to carry out container exchange.

Subsequently, the container holder 62 is elevated by the shifting motor 47 such that the lower end of the cartridge 11 is held to an inserted state in the recovering container 13. Then, the moving head 40 is moved to stop the separate injection nozzle for recovering solution 51r on the first cartridge (C1) to inject the recovering solution R in a predetermined amount. Next, the moving head 40 is moved to the cartridges 11 which are registered in the list of subjects to be treated as a cartridge which is a subject to be treated (in the example shown in FIG. 5, the fourth cartridge (C4)), to carry out injection of the recovering solution R in order. If injection of the recovering solution R is completed for all of the cartridges 11, which are registered in the list of subjects to be treated as a cartridge which is a subject to be treated, supply of pressurized air is conducted for each of the cartridges 11 that are registered in the same manner as described above.

The recovering solution R, for which pressurized air has been supplied in the same manner as described above, and the pressure is given, passed through the nucleic acid-adsorbing porous membrane 11b to desorb nucleic acid which is being adsorbed on this, and nucleic acid is discharged with the recovering solution R into the recovering container 13 from the discharge unit 11c of the lower part. If all of the recovering solution R is discharged into the recovering containers 13 of the cartridges 11, the moving head 40 moves to the shelter position on the immediate above of the first waste liquor container 57 that a series of operations are completed.

The container holder 62 for which the extraction operation has been completed, is lowered by driving the shifting motor 47 to release fitting of the positioning hole 52d of the container holder 62 and the positioning pin 31a of the operation member 31, so the holding device 3 (the cartridge holder 61, the container holder 62 and the container holding part 63) is taken out as combined from the main body of the apparatus 2. Then, cartridge 11 and the waste liquor container 12 are taken out to be discarded from the cartridge holder 61 and the container holder 62. On the other hand, the recovering container 13 is taken out from the container holder 62, and it is capped if necessary, and the next nucleic acid analysis treatment, and the like are carried out.

The air supplied to the cartridge 11 from the air pump 43 may be any gas if it does not affect the properties of a sample solution, a washing solution, a recovering liquid, and the like.

In addition, if a holding device 3 (a cartridge holder 61, a container holder 62 and a container holding part 63) is provided in plural number of groups, it is possible to operate more efficient extraction when the operation of preparation is carried out for the next sample solution S during the operation of the nucleic acid extraction as described above.

Next, a nucleic acid-adsorbing porous solid phase which is provided for the cartridge 11 (herein, nucleic acid-adsorbing porous membrane as an example) will be illustrated in detail.

Herein, the nucleic acid-adsorbing solid phase may be what contains silica or a derivative thereof, diatomite, or alumina. Furthermore, the solid phase may be what contains an organic polymer. The organic polymer is preferably an organic polymer having a polysaccharide structure. In addition, the organic polymer may be acetylcellulose. Furthermore, the organic polymer may be an organic polymer obtained by saponification of a mixture of acetylcelluloses different from each other in acetyl value. The organic polymer may be regenerated cellulose. This will be illustrated in detail below.

The nucleic acid-adsorbing solid phase 11b which is contained in the cartridge 11 is basically porous through which nucleic acid can pass, and it is constituted that the surface thereof has a property to adsorb nucleic acid in a sample solution with chemical binding force, and it maintains the adsorption in washing by a washing solution, and the nucleic acid adsorbing force is weakened in recovering by a recovering solution to be separated.

The nucleic acid-adsorbing solid phase 11b, which is contained in the cartridge 11, is preferably a porous solid phase which adsorbs nucleic acid with an interaction which involves no significant ionic bond. This means that “ionization” does not occur under the usage conditions for the porous solid phase side, and it is presumed that with changing the polarity of the environment, nucleic acid and the porous solid phase are brought to attraction. According to this, it is possible to isolate and purify nucleic acid in excellent separation performance, but also in good washing efficiency. Preferably, nucleic acid-adsorbing porous solid phase is a porous solid phase having a hydrophilic group, and it is presumed that with changing the polarity of the environment, nucleic acid and the porous solid phase are brought to attraction.

The hydrophilic group refers to a polar group (atomic group) which can have interaction with water, and includes all of the groups (atomic groups) which are involved in nucleic acid adsorption. The hydrophilic group has desirably moderate strength of the interaction with water (see “a group which is not too strong in hydrophilicity” in the item of “hydrophilic group,” Dictionary of Chemistry, KYORITSU SHUPPAN CO., LTD.), and for example, it is a hydroxyl group, a carboxyl group, a cyano group, an oxyethylene group, or the like. It is preferably a hydroxyl group.

Herein, the porous solid phase having a hydrophilic group means a porous solid phase wherein the material which forms a porous solid phase has a hydrophilic group as itself, or a porous solid phase wherein a hydrophilic group is introduced by treating or coating a material which forms a porous solid phase. The material which forms a porous solid phase may be an organic substance or an inorganic substance. For example, it is a porous solid phase wherein the material which forms a porous solid phase is an organic material having a hydrophilic group as itself, a porous solid phase wherein a hydrophilic group is introduced by treating a porous solid phase of an organic material having no hydrophilic group, a porous solid phase wherein a hydrophilic group is introduced by coating an organic material having no hydrophilic group with a material having a hydrophilic group, a porous solid phase wherein the material which forms a porous solid phase is an inorganic material having a hydrophilic group as itself, a porous solid phase wherein a hydrophilic group is introduced by treating a porous solid phase of an inorganic material having no hydrophilic group, a porous solid phase wherein a hydrophilic group is introduced by coating an inorganic material having no hydrophilic group with a material having a hydrophilic group, and the like. However, in terms of easy processing, an organic material such as an organic polymer is preferably used for the material which forms a porous solid phase.

The porous solid phase of a material having a hydrophilic group is, for example, a porous solid phase of an organic material having a hydroxyl group. The porous solid phase of an organic material having a hydroxyl group includes a porous solid phase which is formed with polyhydroxyethylacrylic acid, polyhydroxyethylmethacrylic acid, polyvinyl alcohol, polyvinyl pyrrolidone, polyacrylic acid, polymethacrylic acid, polyoxyethylene, acetylcellulose, a mixture of acetylcelluloses different from each other in acetyl value. Particularly, a porous solid phase of an organic material having a polysaccharide structure can be preferably used.

The porous solid phase of an organic material having a hydroxyl group is preferably a porous solid phase of an organic material which comprises a mixture of acetylcelluloses different from each other in acetyl value. The mixture of acetylcelluloses different from each other in acetyl value is preferably a mixture of triacetylcellulose and diacetylcellulose, a mixture of triacetylcellulose and monoacetylcellulose, a mixture of triacetylcellulose, diacetylcellulose and monoacetylcellulose, a mixture of diacetylcellulose and monoacetylcellulose. Particularly, it is preferably a mixture of triacetylcellulose and diacetylcellulose. The mixing ratio (mass ratio) of triacetylcellulose and diacetylcellulose is preferably 99:1 to 1:99, and more preferably 90:10 to 50:50.

A more preferred organic material having hydroxyl groups may be exemplified by the surface saponification products of acetylcellulose described in JP-A No. 2003-128691. The surface saponification product of acetylcellulose is a product obtained by saponifying a mixture of acetylcelluloses different from each other in acetyl value, and the saponification product of a mixture of triacetylcellulose and diacetylcellulose, the saponification product of a mixture of triacetylcellulose and monoacetylcellulose, the saponification product of a mixture of triacetylcellulose, diacetylcellulose and monoacetylcellulose, and the saponification product of a mixture of diacetylcellulose and monoacetylcellulose can be preferably used. More preferably, the saponification product of a mixture of triacetylcellulose and diacetylcellulose are used. The mixing ratio (mass ratio) of a mixture of triacetylcellulose and diacetylcellulose is preferably 99:1 to 1:99. More preferably, the mixing ratio of a mixture of triacetylcellulose and diacetylcellulose is 90:10 to 50:50. In this case, the degree of saponification treatment (rate of saponification) can be controlled by the amount (density) of the hydroxyl groups on the solid phase surface. In order to increase the separation efficiency of nucleic acid, it is preferable that the amount (density) of hydroxyl groups is large. For example, in the case of acetylcelluloses such as triacetylcellulose, the rate of saponification (rate of surface saponification) is preferably about 5% or greater, and more preferably 10% or greater. Furthermore, in order to increase the surface area of the organic high polymer having hydroxyl groups, the porous solid phase of acetylcellulose is preferably subjected to saponification. In this case, the porous solid phase may be a porous membrane having symmetry in the surface and the interior, but a porous membrane having dissymmetry in the surface and the interior can be preferably used.

The treatment of saponification refers to the contacting of acetylcellulose with a solution for saponification treatment (for example, an aqueous solution of sodium hydroxide). Thus, the portion of the acetylcellulose contacted with the solution for saponification treatment is changed to regenerated cellulose, where hydroxyl groups are introduced. The regenerated cellulose thus produced is different from the original cellulose in the crystalline state or the like.

Further, in order to change the rate of saponification, it is preferable to carry out the saponification treatment by changing the concentration of sodium hydroxide. The rate of saponification can be easily measured by NMR, IR or XPS (for example, the rate of saponification can be determined by the degree of peak reduction for a carbonyl group).

As the method for introducing hydrophilic groups to a porous solid phase of an organic material having no hydrophilic groups, graft polymer chains having hydrophilic groups in the polymer chains or in the side chains can be bound to the porous solid phase.

As the method for binding graft polymer chains to a porous solid phase of organic material, mention may be made of two methods including a method for chemically binding graft polymer chains to the porous solid phase, and a method for polymerizing a compound having a polymerizable double bond, with the porous solid phase used as the starting point, to obtain graft polymer chains.

First, in the method for attaching the porous solid phase and the graft polymer chains by chemical binding, the polymer chains can be grafted by using a polymer having a functional group which is reactive with the porous solid phase, at the terminals or in the side chains of the polymer, and chemically reacting this functional group of the polymer with the functional group of the porous solid phase. The functional group which is reactive with the porous solid phase is not particularly limited as long as it can react with the functional group of the porous solid phase, but examples thereof include a silane coupling group such as alkoxysilane, an isocyanate group, an amino group, a hydroxyl group, a carboxyl group, a sulfonic acid group, a phosphoric acid group, an epoxy group, an allyl group, a methacryloyl group, an acryloyl group and the like.

A particularly useful compound as the polymer having a reactive functional group at the terminals or in the side chains of the polymer may be exemplified by a polymer having trialkoxysilyl groups at the polymer terminals, a polymer having amino groups at the polymer terminals, a polymer having carboxyl groups at the polymer terminals, a polymer having epoxy groups at the polymer terminals and a polymer having isocyanate groups at the polymer terminals. The polymer used for this purpose is not particularly limited as long as it has hydrophilic groups that are involved with the adsorption of nucleic acid, but specific examples thereof include polyhydroxyethylacrylic acid and polyhydroxyethylmethacrylic acid and salts thereof, polyvinyl alcohol, polyvinylpyrrolidone, polyacrylic acid and polymethacrylic acid and salts thereof, polyoxyethylene and the like.

The method for forming graft polymer chains by polymerizing a compound having a polymerizable double bond, with the porous solid phase used as the starting point, is generally referred to as surface graft polymerization. The method for surface graft polymerization refers to a method for imparting an active species on the substrate surface by means of plasma irradiation, photoirradiation, heating or the like, and binding a compound having a polymerizable double bond that is disposed to be in contact with the porous solid phase, to the porous solid phase by polymerization.

A compound which is useful for forming graft polymer chains bound to the substrate is required to have two features such as one of having a polymerizable double bond, and the other of having a hydrophilic group that is involved with the adsorption of nucleic acid. For such compound, any compound among polymers, oligomers and monomers having hydrophilic groups can be used, as long as the compound has a double bond in the molecule. A particularly useful compound is a monomer having a hydrophilic group.

Specific examples of the particularly useful monomer having a hydrophilic group include the following monomers. For example, monomers containing hydroxyl-like groups such as 2-hydroxyethylacrylate, 2-hydroxyethylmethacrylate, glycerol monomethacrylate and the like can be particularly preferably used. Carboxyl group-containing monomers such as acrylic acid, methacrylic acid and the like, or alkali metal salts and amine salts thereof also can be preferably used.

As a different method for introducing hydrophilic groups to the porous solid phase of an organic material having no hydrophilic groups, a material having hydrophilic groups can be coated. The material to be used for the coating is not particularly limited as long as the material has hydrophilic groups that are involved with the adsorption of nucleic acid, but from the viewpoint of ease of operation, polymers of organic material are preferred. Examples of the polymers include polyhydroxyethylacrylic acid and polyhydroxyethylmethacrylic acid and salts thereof, polyvinyl alcohol, polyvinylpyrrolidone, polyacrylic acid and polymethacrylic acid and salts thereof, polyoxyethylene, acetylcellulose, mixtures of acetylcelluloses different from each other in acetyl value, and the like, and a polymer having a polysaccharide structure is preferred.

In addition, a porous solid phase of an organic material having no hydrophilic group can be coated with acetylcellulose or a mixture of acetylcelluloses different from each other in acetyl value, and then the coated acetylcellulose or the mixture of acetylcelluloses different from each other in acetyl value can be subjected to saponification. In this case, the rate of saponification is preferably about 5% or greater. Moreover, the rate of saponification is more preferably about 10% or greater.

The porous solid phase which is an inorganic material having hydrophilic groups may be exemplified by porous solid phases containing silica or a derivative thereof, diatomaceous earth or alumina as described above. The porous solid phase containing a silica compound may be exemplified by glass filter. Further, mention may be made of the porous silica thin membrane as described in Japanese Patent No. 3058342. This porous silica thin membrane can be produced by spreading on a substrate, a spreading solution containing a cationic amphiphilic material capable of forming a bimolecular layer, subsequently conditioning the multilayered thin membrane of bimolecular layer of the amphiphilic material by removing the solvent from the liquid membrane on the substrate, contacting the multilayered thin membrane of bimolecular layer with a solution containing a silica compound, and then removing by extraction the multilayered thin membrane of bimolecular layer.

As the method for introducing hydrophilic groups to a porous solid phase of an inorganic material having no hydrophilic group, mention may be made of two methods including a method for chemically binding the porous solid phase with graft polymer chains, and a method for polymerizing graft polymer chains using a monomer having a hydrophilic group which has a double bond in the molecule and, with the porous solid phase used as the starting point.

In the case of attaching the porous solid phase with the graft polymer chains by chemical binding, a functional group which is reactive with the functional group at the terminal of the graft polymer chains is introduced to the inorganic material, and the graft polymer is chemically bound to the inorganic material. In the case of polymerizing graft polymer chains using a monomer having a hydrophilic group which has a double bond in the molecule, with the porous solid phase used as the starting point, a functional group which serves as the starting point for the polymerization of the compound having double bond is introduced into the inorganic material.

As the graft polymer having hydrophilic groups and the monomer having a hydrophilic group which has a double bond in the molecule, those graft polymers having hydrophilic groups and those monomers having a hydrophilic group which have a double bond in the molecule described for the above-described method for chemically binding the porous solid phase of an organic material having no hydrophilic groups and graft polymer chains can be preferably used.

As a different method for introducing hydrophilic groups to the porous solid phase of an inorganic material having no hydrophilic groups, a material having hydrophilic groups can be coated. The material to be used for the coating is not particularly limited as long as the material has hydrophilic groups that are involved with the adsorption of nucleic acid, but from the viewpoint of ease of operation, polymers of organic material are preferred. Examples of the polymers include polyhydroxyethylacrylic acid and polyhydroxyethylmethacrylic acid and salts thereof, polyvinyl alcohol, polyvinylpyrrolidone, polyacrylic acid and polymethacrylic acid and salts thereof, polyoxyethylene, acetylcellulose, mixtures of acetylcelluloses different from each other in acetyl value, and the like, and a polymer having a polysaccharide structure is preferred.

In addition, a porous solid phase of an inorganic material having no hydrophilic groups can be coated with acetylcellulose or a mixture of acetylcelluloses different from each other in acetyl value, and then the coated acetylcellulose or the mixture of acetylcelluloses different from each other in acetyl value can be subjected to saponification. In this case, the rate of saponification is preferably about 5% or greater. Moreover, the rate of saponification is more preferably about 10% or greater.

As the porous solid phase of an inorganic material having no hydrophilic groups, mention may be made of porous solid phases prepared by processing metals such as aluminum, glass, cement, ceramics such as porcelain, or new ceramics, silicon, activated carbon or the like.

The nucleic acid-adsorbing porous membrane may be in any form of a porous membrane, non-woven fabric or textile. The nucleic acid-adsorbing porous membrane is capable of permitting a solution to pass through the interior, and thus the thickness of the membrane is 10 μm to 500 μm. More preferably, the thickness is 50 μm to 250 μm. In view of the ease of washing, a membrane having a smaller thickness is more desirable.

The nucleic acid-adsorbing porous membrane which is capable of permitting a solution to pass through the interior has a minimum pore size of 0.22 μm or greater. More preferably, the minimum pore size is 0.5 μm or greater. Further, it is desirable to use a porous membrane having a ratio of the maximum pore size and the minimum pore size of 2 or greater. Then, it is possible to obtain a sufficient surface area for the nucleic acid to adsorb thereon, while it is not easily plugged. Even more preferably, the ratio of the maximum pore size and the minimum pore size is 5 or greater.

The nucleic acid-adsorbing porous membrane which is capable of permitting a solution to pass through the interior has a porosity of 50 to 95%. More preferably, the porosity is 65 to 80%. The bubble point is preferably from 0.1 to 10 kgf/cm². More preferably, the bubble point is from 0.2 to 4 kgf/cm².

The nucleic acid-adsorbing porous membrane which is capable of permitting a solution to pass through the interior preferably has a pressure loss of 0.1 to 100 kPa. Accordingly, uniform pressure can be obtained upon the occurrence of overpressure. More preferably, the pressure loss is from 0.5 to 50 kPa. Here, the pressure loss refers to the minimum pressure required from the membrane to allow water to pass through per 100 μm of the membrane thickness.

For the nucleic acid-adsorbing porous membrane which is capable of permitting a solution to pass through the interior, the amount of water permeated when water is passed through at 25° C. and at a pressure of 1 kg/cm² is preferably 1 to 5000 mL per minute per 1 cm² of the membrane. More preferably, the amount of water permeated when water is passed through at 25° C. and at a pressure of 1 kg/cm² is preferably 5 to 1000 mL per minute per 1 cm² of the membrane.

For the nucleic acid-adsorbing porous membrane which is capable of permitting a solution to pass through the interior, the amount of nucleic acid adsorbed per 1 mg of the porous membrane is preferably 0.1 μg or greater. More preferably, the amount of nucleic acid adsorbed per 1 mg of the porous membrane is 0.9 μg or greater.

The nucleic acid-adsorbing porous membrane which is capable of permitting a solution to pass through the interior is preferably a cellulose derivative which does not dissolve within 1 hour but dissolves within 48 hours, when a square-shaped porous membrane with its each side being 5 mm is immersed in 5 mL of trifluoroacetic acid. Further, more preferred is a cellulose derivative which dissolves within 1 hour when a square-shaped porous membrane with its each side being 5 mm is immersed in 5 mL of trifluoroacetic acid, but which does not dissolve within 24 hours when the same specimen is immersed in 5 mL of dichloromethane.

When a sample solution containing nucleic acid is passed through the nucleic acid-adsorbing porous membrane, it is preferable to pass the sample solution from one side to the other side, from the perspective that the membrane can contact the sample solution with the porous membrane uniformly. When a sample solution containing nucleic acid is passed through the nucleic acid-adsorbing porous membrane, it is preferable to pass through the sample solution from the side having a larger pore size of the porous membrane to the side having a smaller pore size, from the perspective that the membrane is not easily plugged.

When a sample solution containing nucleic acid is passed through the nucleic acid-adsorbing porous membrane, the flow rate is preferably from 2 to 1500 μL/sec per cm² of the surface area of the membrane, in order to provide an appropriate contact time for the solution with the porous membrane. When the contact time for the solution with the porous membrane is excessively short, a sufficient effect of nucleic acid extraction cannot be obtained. When the contact time is excessively long, it is not desirable from the viewpoint of operability. Furthermore, the flow rate is preferably from 5 to 700 μL/sec per cm² of the surface area of the membrane.

A single nucleic acid-adsorbing porous membrane which is capable of permitting the solution used to pass through the interior may be used, but a plurality of such membranes may be also used. The plural nucleic acid-adsorbing porous membranes may be identical or different.

The plural nucleic acid-adsorbing porous membrane may be composed of a combination of nucleic acid-adsorbing porous membranes of an inorganic material and nucleic acid-adsorbing porous membranes of an organic material. For example, mention may be made of a combination of a glass filter and a porous membrane of regenerated cellulose. Further, the plural nucleic acid-adsorbing porous membranes may be composed of a combination of nucleic acid-adsorbing porous membranes of an inorganic material and non-nucleic acid-adsorbing porous membranes of an organic material, and may be exemplified by a combination of a glass filter and a porous membrane of nylon or polysulfone.

Next, the sample solution will be described in detail.

<Sample Solution Containing Nucleic Acid>

The sample solution containing nucleic acid can be obtained by treating a pretreatment solution containing at least one selected from a nucleic acid stabilizer, a chaotropic salt, a surfactant, a buffer, a defoaming agent and a proteolytic enzyme, with a nucleic acid solubilizing reagent, and particularly preferably the solution is a solution obtained by adding a water-soluble organic solvent.

(Test Sample)

The test sample that can be used in the invention is not particularly limited as long as the test sample contains nucleic acid. For example, mention may be made of body fluids such as collected whole blood, blood plasma, blood serum, urine, faeces, semen, saliva or the like in the field of diagnosis, a compound derived from a living body such as animals (or parts thereof), or biological materials such as plants (or parts thereof), bacteria, viruses or the like. The test sample may be used as received, or a dissolution liquid or homogenate thereof may be also used as the sample.

The “sample” means any sample containing nucleic acid. More specifically, mention may be made of those described with respect to the above-described test samples. There may be one type of the nucleic acid, or two or more types of the nucleic acid in the sample solution. The length of each nucleic acid that is provided to the above-described method for separating and purifying nucleic acid is not particularly limited, and for example, a nucleic acid of any length from a few bps to a few Mbps. In general, from the viewpoint of handlability, the length of the nucleic acid is preferably in the range of a few bps to a few hundred kbps.

According to the invention, the “nucleic acid” may be any DNA or RNA of a single strand or double strand, and the molecular weight thereof is also not limited.

The test sample can be preferably obtained as a sample solution containing nucleic acid by solubilizing the cell membrane, nuclear membrane and the like, and dispersing the nucleic acid in an aqueous solution.

### EXAMPLE 1

An example will be explained below wherein the presence or absence of the cartridge which is a subject to be treated was ascertained by the nucleic acid extracting apparatus as described above, and for the cartridge which was discriminated as a subject to be treated, nucleic acid extraction was conducted.

(1) Preparation of Container for Nucleic Acid Separation and Purification

A cartridge (a container for nucleic acid separation and purification), which has the inner diameter of 7 mm and contains a solid phase for nucleic acid adsorption, was prepared from polypropylene.

(2) Nucleic Acid Separating and Purifying Apparatus

Acetylcellulose porous membrane was used as a nucleic acid-adsorbing porous membrane, and was placed in the nucleic acid-adsorbing porous membrane holder part in the cartridge for nucleic acid purification prepared in (1) above. The porous membrane having the average pore size of 2 μm is used.

(3) Preparation of DNA Solubilizing Reagent and Washing Solution

The DNA solubilizing reagent and washing solution as prescribed in Table 1 were prepared.

TABLE 1

| | | | |
|---|---|---|---|
| DNA solubilizing | Guanidine | 382 | g |
| reagent | hydrochloride | | |
| | (manufactured by | | |
| | Life Technologies | | |
| | Co., Ltd.) | | |
| | Tris (manufactured | 12.1 | g |
| | by Life | | |
| | Technologies Co., | | |
| | Ltd.) | | |
| | TritonX-100 | 10 | g |
| | (manufactured by | | |
| | ICN) | | |
| | Distilled water | 1000 | ml |

| | |
|---|---|
| Washing solution | 10 mM Tris-HCl 50% ethanol |

(4) Nucleic Acid Purification Operation

5 μg of λDNA (manufactured by Clontech Laboratories, Inc.) was dissolved in 100 μl of TE buffer, which was taken as an aqueous DNA solution. To this solution, 100 μl of the DNA solubilizing reagent of the formulation shown in Table 1 was added, and the mixture was stirred.

After stirring, 800 μl of ethanol with the various concentrations shown in Table 2 was added thereto and the mixture was stirred. Then, the nucleic acid particle of the nucleic acid-containing reagent, which was treated as described above, was measured for particle size thereof with a dynamic light scattering spectrometer (DLS7000). The results of the measurement were shown in Table 3.

TABLE 2

| | Level 1 | Level 2 | Level 3 | Level 4 |
|---|---|---|---|---|
| Ethanol | 50% | 70% | 90% | 100% |
| Concentration | | | | |

TABLE 3

| | Level 1 | Level 2 | Level 3 | Level 4 |
|---|---|---|---|---|
| Nucleic | 0.05 μm | 0.13 μm | 1.1 μm | 2.1 μm |
| acid | | | | |
| particle | | | | |
| size | | | | |

After the measurement, the nucleic acid-containing sample, which has been treated as described above, was injected into a cartridge having a nucleic acid-adsorbing porous membrane of an organic polymer composed of the mixture of the acetylcelluloses prepared in the above (1) and (2). Subsequently, the cartridge was connected to a pressurized air supply device to supply pressurized air, and thereby make the inside of the cartridge as pressed state. The injected sample solution containing nucleic acid-containing sample is passed through the nucleic acid-adsorbing porous membrane, and thereby brought into contact with the nucleic acid-adsorbing porous membrane, and discharged from the cartridge. Subsequently, the washing solution shown in Table 1 was injected into the cartridge, and pressurized air was supplied from the pressurized air supply device to pressurize the cartridge in the same manner as the above. The injected washing solution was passed through the nucleic acid-adsorbing porous membrane, discharged and washed. Subsequently, a recovering solution was injected into the cartridge, and pressurized air was supplied from the pressurized air supply device to pressurize the cartridge in the same manner as the above. The injected recovering solution was passed through the nucleic acid-adsorbing porous membrane to be discharged, and this solution was recovered in a recovering container.

(5) Conformation of DNA Separation and Purification

An absorption spectrum of the recovering solution at 260 nm was measured to determine the yield of DNA. The results of the measurement were shown in Table 4. The liquid passage time at this time was shown in Table 5.

TABLE 4

| | Level 1 | Level 2 | Level 3 | Level 4 |
|---|---|---|---|---|
| yield of | 4.3 μg | 4.1 μg | 1.3 μg | 0.2 μg |
| DNA | | | | |

TABLE 5

| | Level 1 | Level 2 | Level 3 | Level 4 |
|---|---|---|---|---|
| Liquid | 8 seconds | 11 seconds | 450 | 2100 |
| passage | | | seconds | seconds |
| time | | | | |

Furthermore, in the above embodiment, the apparatus for recovering a specific substance has been explained as a nucleic acid extracting apparatus, but it can be a protein extracting apparatus by changing the cartridge as a protein extraction cartridge for extracting proteins.

### INDUSTRIAL APPLICABILITY

According to the apparatus for recovering a specific substance and a nucleic acid extracting apparatus using the same of the present invention, it is possible to conduct recovery treatment for a specific substance (nucleic acid extracting treatment, etc.) in high efficiency, simply and quickly, and in excellent automation suitability.

The entire disclosure of each and every foreign patent application from which the benefit of foreign priority has been claimed in the present application is incorporated herein by reference, as if fully set forth.

1. An apparatus for recovering a specific substance in a sample solution, which comprises:
a cartridge holder that can contain a plurality of cartridges; and
a plurality of cartridges each having a filter member, and held by the cartridge holder,
wherein a sample solution is injected into the cartridge held by the cartridge holder, and a pressure is applied to adsorb a specific substance in the sample solution on the filter member, and a recovering solution is injected into the cartridge, and a pressure is applied to recover the specific substance adsorbed on the filter member with the recovering solution, and
wherein the apparatus further comprises:
a pressurized air supply means that introduces a pressurized air from a pressurizing nozzle into the cartridge;
a pressure detecting means that detects a pressure within the cartridge; and
a means for deciding a subject to be treated that decides whether or not the cartridge is a cartridge which is a subject to be treated for recovering the specific substance, based on the pressure detected by the pressure detecting means when the pressurized air is introduced into the cartridge by the pressurized air supply means.

2. The apparatus for recovering a specific substance in a sample solution according to claim 1, wherein the means for deciding a subject to be treated excludes a cartridge from the subject to be treated when a peak value of the pressure within the cartridge, which is detected by the pressure detection means, is lower than a preset value.

3. The apparatus for recovering a specific substance in a sample solution according to claim 1,
wherein the means for deciding a subject to be treated excludes a cartridge from the subject to be treated when an integral value for a certain time of the pressure within the cartridge, which is detected by the pressure detection means, is lower than a preset value.

4. The apparatus for recovering a specific substance in a sample solution according to claim 1,
wherein the pressurizing nozzle of the pressurized air supply means is supported movably along a direction of a cartridge mounting of the plurality of cartridges contained in the cartridge holder.

5. The apparatus for recovering a specific substance in a sample solution according to claim 4,
wherein the pressurizing nozzle and at least a separate injection nozzle that discharges the recovering solution, are installed as integrated with a movable body.

6. The apparatus for recovering a specific substance in a sample solution according to claim 1,
wherein the filter member is one of a porous membrane, a non-woven fabric and a textile.

7. The apparatus for recovering a specific substance in a sample solution according to claim 1,
wherein the specific substance is a living body-derived substance or a biological material.

8. An apparatus for recovering a specific substance in a sample solution according to claim 1, which is a nucleic acid extracting apparatus,
wherein the cartridge having a filter member is a nucleic acid extraction cartridge for extracting nucleic acid, and the specific substance is nucleic acid.
